(19) United States
(12) Patent Application Publication
(10) Pub. No.: US 2020/0229781 A1
(43) Pub. Date: Jul. 23, 2020

(54) X-RAY IMAGING APPARATUS AND METHOD FOR DETERMINING INSTALL LOCATION OF DETECTOR

(71) Applicant: Samsung Electronics Co., Ltd., Suwon-si, Gyeonggi-do (KR)

(72) Inventor: Seung Hoon LIM, Suwon-si (KR)

(73) Assignee: Samsung Electronics Co., Ltd., Suwon-si, Gyeonggi-do (KR)

(21) Appl. No.: 16/650,088

(22) PCT Filed: Sep. 3, 2018
(86) PCT No.: PCT/KR2018/010224

§ 371 Date: Mar. 24, 2020

(30) Foreign Application Priority Data
Sep. 29, 2017 (KR) 10-2017-0126899

(51) Int. Cl. A61B 6/00; G01N 23/04

(52) U.S. Cl. CPC A61B 6/465; A61B 6/467; A61B 6/463; A61B 6/4266; G01N 2223/408; G01N 23/04; A61B 6/4464; A61B 6/4405; A61B 6/54

(57) ABSTRACT

Disclosed are an X-ray imaging apparatus and a method for determining a position of a detector. in accordance with an aspect of the present embodiment, an X-ray imaging apparatus includes an input receiving a user command; an X-ray source configured to radiate X-rays onto a subject according to the user command; a plurality of detectors respectively outputting a plurality of images in response to performing an X-ray radiation operation of the X-ray source; a controller configured to determine at least one image among the plurality of images, wherein the at least one image includes an image corresponding to a detector from which the X-ray is estimated to be incident from the plurality of detectors; and a display configured to display the at least one image.

### CROSS-REFERENCE TO RELATED APPLICATIONS

This application is a U.S. National Stage Application which claims the benefit under 35 USC 371 of International Patent Application No. PCT/KR2018/010224 filed Sep. 3, 2018, which claims foreign priority benefit under 35 USC 119 of Korean Patent Application No. 10-2017-0126899 filed Sep. 29, 2017 in the Korean Intellectual Property office, the contents of both of which are incorporated herein by reference.

### TECHNICAL FIELD

Embodiments of the disclosure relate to an X-ray imaging apparatus and a method for determining an install location of a detector.

### BACKGROUND ART

An X-ray imaging apparatus obtains an image of the inside of a subject by radiating X-rays to the subject, and displays the obtained image to allow a user (e.g., a doctor, a nurse, a clinical pathologist, a radiologist, a patient, or a security person) to visually identify tissues, structures, or objects inside the subject. Here, the subject may include, for example, a human body, a plant or luggage.

The X-ray imaging apparatus may acquire the image of the inside of the subject by using X-rays absorbed or transmitted through a material depending on the properties of the material inside the subject, for example, the density. For example, a conventional X-ray imaging apparatus irradiates X-rays to all or part of a subject, receives X-rays passing through all or part of the subject, and then converts the received X-rays into corresponding electrical signals, and then obtains an X-ray image based on the corresponding electrical signal.

Such X-ray imaging apparatus may include, for example, a digital X-ray imaging apparatus (DR), a mammography apparatus, and a computed tomography apparatus (CT). These X-ray imaging apparatuses are widely used in various industries, such as the health care industry, the security system industry, and the construction industry.

### DISCLOSURE

### Technical Problem

It is an object of the disclosure to provide an X-ray imaging apparatus including a plurality of detectors that can be arranged at various locations, capable of properly determining a detector disposed at a photographing position among the plurality of detectors and a method for determining an install location of the detector.

### Technical Solution

In order to solve the above problems, there is provided an X-ray imaging apparatus and a determination method of an install location of a detector. In accordance with a present disclosure, an X-ray imaging apparatus includes an input receiving a user command; an X-ray source configured to radiate X-rays onto a subject according to the user command; a plurality of detectors respectively outputting a plurality of images in response to performing an X-ray radiation operation of the X-ray source; and a controller configured to determine at least one image among the plurality of images, wherein the at least one image includes an image corresponding to the detector from which the X-ray is estimated to be incident from the plurality of detectors; and a display configured to display the at least one image.

Further, the controller may detect the image including the subject by analyzing the plurality of images separately, and estimate the detector that transmits a signal for the image including the subject as the detector to which the X-ray is incident.

Further, the input may receive a command for a capturing position of the X-ray, and the controller may obtain information on a direction of the plurality of detectors from each of the plurality of detectors, and estimate the detector whose direction corresponding to the capturing position of the X-ray as the detector which the X-ray is incident among the plurality of detectors.

Further, the controller may estimate the detector to which the X-ray is incident based on usage history of each of the plurality of detectors.

Further, the display may display the plurality of images simultaneously or sequentially, the input may receive a selection command for any one of the plurality of images, and the controller may determine the detector from which the X-ray is estimated to be incident from among the plurality of detectors according to the selection command.

Further, the input may receive at least one of an approval command and a disapproval command for the at least one image.

Further, the display may display another image of the plurality of images instead of the at least one image in response to the input of the disapproval command.

Further, the input may receive a command for a capturing location, and the controller may recognize the determined detector as the detector corresponding to the capturing location in response to the input of the approval command.

Further, the plurality of detectors may detect whether the X-rays are incident and may initiate detection of the X-rays in response to the incident detection of the X-rays.

Further, the controller may transmit an operation preparation command to the plurality of detectors, and the plurality of detectors may transmit a response signal corresponding to the operation preparation command to the controller in response to receiving the operation preparation command, and may prepare for detection of the X-rays.

In accordance with a present disclosure, a method for determining an install location of a detector, the method includes: radiating X-rays onto a subject by an X-ray source; outputting a plurality of images in response to performing an X-ray radiation operation of the X-ray source by a plurality of the detectors; determining at least one image among the plurality of images, wherein the at least one image includes an image corresponding to the detector from which the X-ray is estimated to be incident from the plurality of detectors; and displaying the at least one determined image.

Further, the determining of the at least one image among the plurality of images, wherein the at least one image includes the image corresponding to the detector from which the X-ray is estimated to be incident from the plurality of detectors; may comprise detecting the image including the subject by analyzing the plurality of images separately; and estimating the detector that transmits a signal for the image including the subject as the detector to which the X-ray is incident.

Further, the method may further include receiving a command for a capturing position of the X-ray, and the determining of the at least one image among the plurality of images, wherein the at least one image includes an image corresponding to the detector from which the X-ray is estimated to be incident from the plurality of detectors; may comprise obtaining information on a direction of the plurality of detectors from each of the plurality of detectors; and determining the detector among the plurality of detectors whose direction corresponds to the capturing position of the X-rays.

Further, the determining of the at least one image among the plurality of images, wherein the at least one image includes an image corresponding to the detector from which the X-ray is estimated to be incident from the plurality of detectors; may comprise determining the detector to which the X-ray is incident based on usage history of each of the plurality of detectors.

Further, the determining of the at least one image among the plurality of images, wherein the at least one image includes an image corresponding to the detector from which the X-ray is estimated to be incident from the plurality of detectors; may comprise displaying the plurality of images simultaneously or sequentially, receiving a selection command for any one of the plurality of images, and determining the detector from which the X-ray is estimated to be incident from among the plurality of detectors according to the selection command.

### Advantageous Effects

According to the above-described X-ray imaging apparatus and the method for determining the install location of the detector, when a subject is photographed through the X-ray imaging apparatus using the plurality of detectors that can be disposed at various positions, it is possible to properly determine whether a certain detector among the plurality of available detectors is mounted at a position to be photographed.

According to the above-described X-ray imaging apparatus and the method for determining the install location of the detector, it is possible to determine the detector mounted at the capturing location without any additional devices, thereby simplifying the device design and reducing the manufacturing cost.

Also, according to the above-described X-ray imaging apparatus and the method for determining the install location of the detector, it is possible to prevent erroneous X-ray emissions due to the operation of the detector that does not exist at the capturing location , as a result, inconvenience caused by unnecessary repetitive shooting can be eliminated.

### DESCRIPTION OF DRAWINGS

FIG. 1 is a diagram of an embodiment of an X-ray imaging apparatus.

FIG. 2 is a diagram illustrating an example in which a plurality of detectors are installed on a shooting table and a stand, respectively.

FIG. 3 is a diagram illustrating an example in which a subject is mounted on a shooting table.

FIG. 4 is a diagram illustrating an example of performing X-ray imaging using a stand.

FIG. 5 is a diagram illustrating an example of performing X-ray imaging without a stand and a table.

FIG. 6 is a block diagram of an embodiment of an X-ray imaging apparatus.

FIG. 7 illustrates an example of communicatively connecting an X-ray imaging apparatus and a detector.

FIG. 8 is a diagram illustrating an example of an image acquired from each detector.

FIG. 9 is a diagram illustrating an example of a screen on which a plurality of images acquired from each detector is displayed.

FIG. 10 is a diagram illustrating an example of a screen on which one selected image is displayed.

FIG. 11 is a block diagram of another embodiment of an X-ray imaging apparatus.

FIG. 12 is a diagram illustrating an example of a screen on which an automatically selected image is displayed.

FIG. 13 is a block diagram of another embodiment of an X-ray imaging apparatus.

FIG. 14 is a block diagram of another embodiment of an X-ray imaging apparatus.

FIG. 15 is a diagram illustrating another embodiment of a detector.

FIG. 16 is a flowchart illustrating an embodiment of a method for position determining of a detector.

FIG. 17 is a flowchart illustrating another embodiment of a method for position determining of a detector.

FIG. 18 is a flowchart illustrating another embodiment of a method for position determining of a detector.

FIG. 19 is a flowchart illustrating another embodiment of a method for position determining of a detector.

FIG. 20 is a flowchart illustrating another embodiment of a method for position determining of a detector.

### BEST MODE

### [Mode for Invention]

Like reference numerals refer to like elements throughout the specification unless otherwise specified. As used herein, the term “part” is added and may be implemented in software or hardware. According to an exemplary embodiment, the term ‘part’ may be implemented as one component or one ‘part’ may be implemented as a plurality of components.

Throughout the specification, when an element is referred to as being “connected to” another element, it may be directly or indirectly connected to the other element and the “indirectly connected to” includes being connected to the other element via a wireless communication network.

Also, it is to be understood that the terms “include” and “have” are intended to indicate the existence of elements disclosed in the specification, and are not intended to preclude the possibility that one or more other elements may exist or may be added.

In this specification, the terms “first,” “second,” etc. are used to distinguish one component from other components and, therefore, the components are not limited by the terms.

An expression used in the singular form encompasses the expression of the plural form, unless it has a clearly different meaning in the context.

The reference numerals used in operations are used for descriptive convenience and are not intended to describe the order of operations and the operations may be performed in an order different unless otherwise stated.

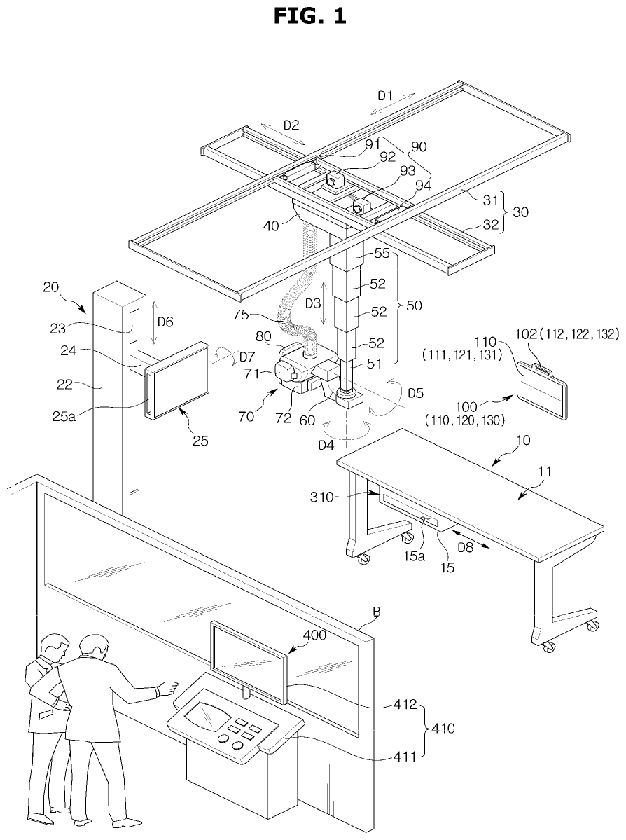

Hereinafter, various embodiments of the X-ray imaging apparatus will be described with reference to FIGS. 1 to 15.

FIG. 1 is a diagram of an embodiment of an X-ray imaging apparatus.

As shown in FIG. 1, an X-ray imaging apparatus 1 may include, in one embodiment, an X-ray source 70 emitting radiation (e.g., X-ray) to the outside, a plurality of detectors 100, and a workstation 400 that provides the overall operation of the X-ray imaging apparatus 1 and provides the user with a predetermined user interface 410.

At least two of the X-ray source 70, the detector 100, and the workstation 400 may communicate with each other based on a wired communication network, a wireless communication network, or a combination thereof The wired communication network may be constructed using a cable, and the cable may be implemented using, for example, a paired cable, a coaxial cable, an optical fiber cable, an Ethernet cable, or the like. The wireless communication network may be implemented using at least one of a short-range communication network and a telecommunication network. Here, the short-range communication network, for example, Wi-Fi, ZigBee, Bluetooth, Wi-Fi Direct, Bluetooth Low Energy, or CAN may be implemented using a communication standard such as infrared data association (IrDA) or near field communication (NFC). The telecommunication network can use mobile communication standards, which include, for example, 3GPP-based wireless communication technologies, such as evolutionary high-speed packet access (HPDA+) or long-term evolution (LTE), and optimized evolution-data. Wireless communication technology such as 3GPP2 series wireless communication technology such as (EV-Do) or WiMAX series such as WiBro evolution may be adopted.

The X-ray source 70 and the detector 100 may be installed inside an X-ray imaging room, and the workstation 400 may be installed in a separate space separated from the X-ray imaging room through a shielding wall B. Accordingly, an operator of the X-ray imaging apparatus 1 may perform X-ray imaging of a subject 9 without being exposed to X-rays generated during the X-ray imaging. Here, the operator of the X-ray imaging apparatus 1 may be a doctor, a nurse, a clinical pathologist, a radiologist or a security person. The X-ray imaging apparatus 1 may be manipulated or may include a variety of persons authorized to manipulate it.

The X-ray source 70 is provided to emit X-rays corresponding to an applied tube voltage and tube current to the outside according to a control signal transmitted from the workstation 400. In this case, the intensity of X-rays radiated from the X-ray source 70 corresponds to the tube voltage, and the dose of X-rays corresponds to the product of the tube current and time. The tube voltage and tube current may be provided from a separate power source (not shown).

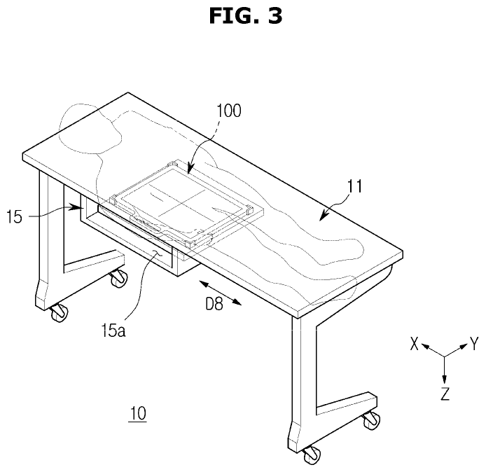

The X-ray source 70 may adjust at least one of an X-ray emission position and an X-ray emission direction so that X-rays may be appropriately emitted onto a subject (9 in FIGS. 3 to 5). Here, the subject 9 may include, for example, a human body, an animal or a plant, baggage, a building, or the like. In addition, the subject 9 may include various living or non-living objects that can be photographed by X-rays.

According to an embodiment, as shown in FIG. 1, the X-ray imaging apparatus 1 may include a guide rail 30 and a moving carriage 40, and a post frame 50 for adjusting at least one of a position and an X-ray radiation direction of the X-ray source 70.

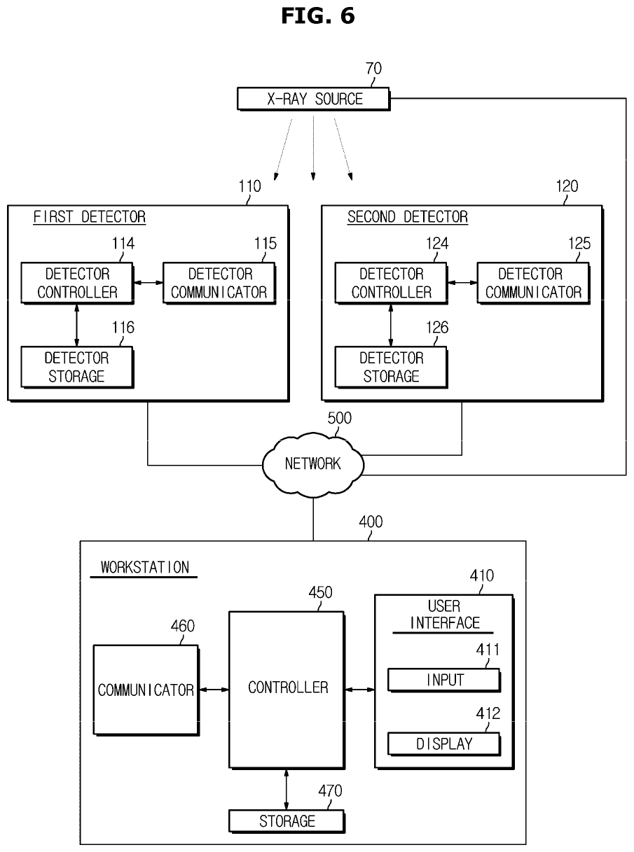

The X-ray imaging apparatus 1 may further include a driver 90 (91 to 94) for moving each of a second guide rail 32 and the moving carriage 40. The driver 90 may be implemented using a roller and a motor, and according to an exemplary embodiment, the driver 90 may be implemented using various devices capable of moving an object, such as an actuator. The driver 90 starts driving according to a control signal of a controller 450 of FIGS. 6, 11, and 14. The position of the X-ray source 70 may be moved by applying a rotational force in a predetermined direction to the component connected to the driver 90.

The guide rail 30 may be installed at one position inside the X-ray imaging room, and may be installed at, for example, a ceiling of the X-ray imaging room. The guide rail 30 may include a first guide rail 31 and the second guide rail 32 provided to cross each other at a predetermined angle. In this case, the predetermined angle may be 90 degrees. In other words, the first guide rail 31 and the second guide rail 32 may be perpendicular to each other. The first guide rail 31 is directly installed on the bottom surface of the ceiling of the X-ray imaging room, and the second guide rail 32 is disposed in a predetermined direction dl along the first guide rail 31 below the first guide rail 31. It can be mounted so as to reciprocate. According to an embodiment, at least one of the drivers 91 or 94 which may be rotatable may be provided between the first guide rail 31 and the second guide rail 32. The second guide rail 32 may be slidably moved along the first guide rail 31 by the rotation of rollers or the like provided in the drivers 91 and 94.

The moving carriage 40 is disposed below the second guide rail 32 and is provided to be movable in a predetermined direction d2 along the second guide rail 32. According to one embodiment, the moving carriage 40 may be provided with at least one of the drivers 92 and 93 to rotate and travel along the second guide rail 32, so that the moving carriage 40 can be slidably moved in the direction d2 of the second guide rail 32 different from the movable direction dl of the second guide rail 32. The different direction d2 may include a direction orthogonal to the movable direction dl of the second guide rail 32.

The post frame 50 may have a plurality of posts 51, 52, 53, 54, and 55, one end of which is mounted below the moving carriage 40. The plurality of posts 51, 52, 53, 54, and 55 may be combined such that one can be inserted into the other. In this case, at least one of the plurality of posts 51, 52, 53, 54, and 55 is inserted into the other, so that the post frame 50 can be shortened and/or extended while being fixed to the moving carriage 40. Accordingly, the X-ray source 70 may move in at least one direction d3 of the up direction and the down direction. In other words, the vertical position of the X-ray source 70 can be adjusted. Specifically, when the post frame 50 is shortened, the X-ray source 70 moves in the ceiling direction of the X-ray examination room. When the post frame 50 is extended, the X-ray source 70 moves in the bottom direction of the X-ray examination room.

The other end of the post frame 50 may be equipped with a rotary joint 60. The rotary joint 60 is rotatably provided in at least one rotation direction d4 so that the front surface of the X-ray source 70 can face a predetermined direction while coupling the X-ray source 70 to the post frame 50. Here, the predetermined direction may include a direction orthogonal to the direction d3 in which the X-ray source 70 is movable by the post frame 50. A driver (not shown) may be provided between the distal end of the post 51 disposed at the bottom and the rotary joint 60, whereby the rotary joint 60 may be rotated under the control of the controller 450. As the rotary joint 60 rotates, the X-ray source 70 also rotates in the predetermined direction d4 about an axis extending in the vertical direction.

The X-ray source 70 may be attached to one direction of the rotary joint 60. The X-ray source 70 may move in the various directions dl to d3 according to the movement of the second guide rail 32 and/or the moving carriage 40 or the extension and contraction of the post frame 50. It may also be panned or tilted in accordance with the rotation of the rotary joint 60 in the predetermined directions dl and d4. Accordingly, the X-ray source 70 may emit X-rays at various locations and/or in various directions. The X-ray source 70 is associated with an X-ray tube 71 for generating and emitting X-rays according to an applied electrical signal, a collimator 72 for adjusting the emission range of the emitted X-rays, and/or the emission position of the X-rays, and X-ray emission, and it may include various circuit boards, conductive wires, etc. for performing various electrical signal transfers. The X-ray tube 71, the collimator 72, and substrate or the conductive wires are mounted and installed inside the housing for implementing the X-ray source 70. The X-ray source 70 may further include a cover 75 in which a cable for transmitting a current supplied from the outside is embedded.

According to an embodiment, an auxiliary user interface device 80 may be further provided on the side of the outer housing of the X-ray source 70 to provide information to the user and to receive a control command from the user. The auxiliary user interface device 80 may include an auxiliary input (not shown, for example, a button, a knob, a trackball, a touch pad, etc.) capable of receiving predetermined commands or data related to the operation of the X-ray source 70, and/or an auxiliary display (not shown) for providing various types of information. According to an exemplary embodiment, an auxiliary display unit 81 may be implemented as a touch screen.

The detector 100 is provided to be portable by the user. That is, the detector 100 may be a portable detector. In this case, the detector 100 is physically separated from a table 10 and a stand 20 and the like, but is provided to be mounted or detached from the table 10 and/or the stand 20.

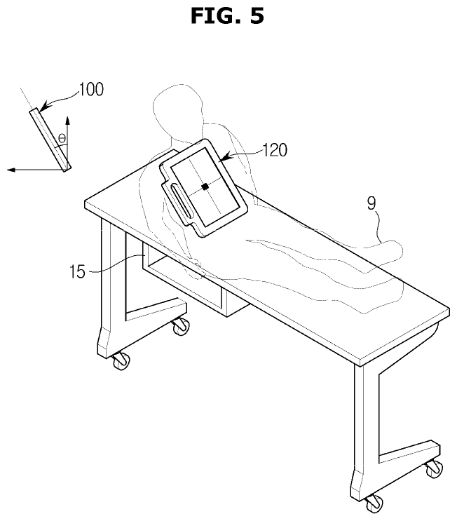

The detector 100 may have an incident surface 101 through which an X-ray passing through an object is incident, and a sensing panel (not shown) for detecting the incident X-rays may be installed inside the detector 100. The sensing panel may be provided at a position corresponding to the incident surface 101 inside the detector 100. A handle 111 may be provided at the center of the upper end of the detector 100 for the convenience of the user. In addition, a storage battery 100a of FIG. 5 may be further installed inside the detector 100 to supply power to various devices provided in the detector 100 such as a sensing panel.

According to an exemplary embodiment, the sensing panel may use a direct conversion method or an indirect conversion method to convert X-rays into electrical signals. In the case of a sensing panel employing a direct conversion method, for example, X-rays may be converted into electrical signals by electrically converting a movement of an electron-hole pair corresponding to incident X-rays. In the case of a sensing panel employing an indirect conversion method, X-rays may be converted into electrical signals by converting the X-rays into visible rays using a scintillator and converting the converted visible rays into electrical signals using a photodiode or the like.

In addition, the sensing panel may be implemented by employing a charge integration mode for storing a charge for a predetermined time and then obtaining a signal therefrom. Alternatively, a photon counting mode may be implemented by counting whenever a signal having a threshold value or more is generated by a single X-ray photon.

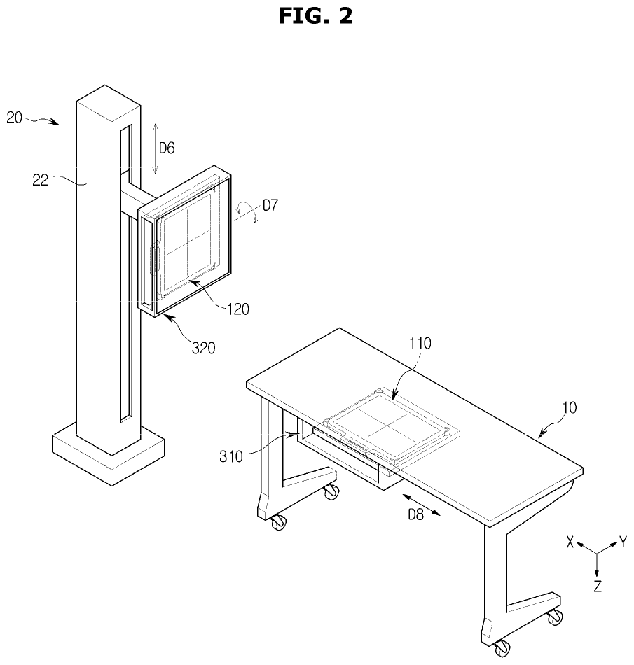

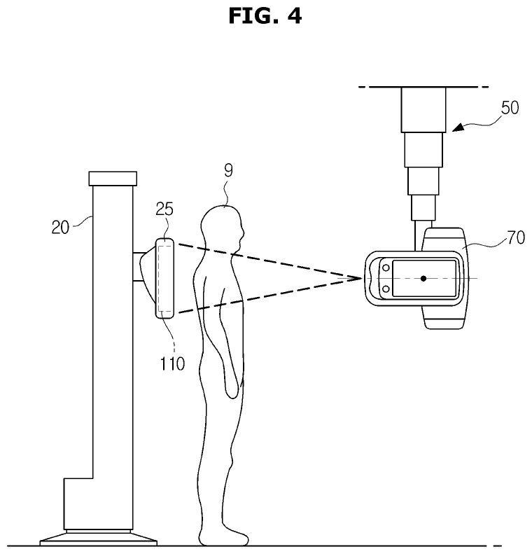

FIG. 2 is a diagram illustrating an example in which a plurality of detectors are installed on a shooting table and a stand, respectively, FIG. 3 is a diagram illustrating an example in which a subject is mounted on a shooting table, FIG. 4 is a diagram illustrating an example of performing X-ray imaging using a stand, and FIG. 5 is a diagram illustrating an example of performing X-ray imaging without a stand and a table.

Referring to FIGS. 1 to 3, the table 10 is provided so that all or a part of the subject 9 may be mounted, and a mounting surface 11 provided substantially horizontally with respect to the floor surface (or ground), and a table mounting portion 15 is formed so that the detector 100 can be inserted and mounted on the top, middle or bottom of the mounting surface 11.

The table mounting portion 15 may be provided, for example, in a state parallel to the bottom surface, that is, in a state parallel to the plane formed by the x and y axes. The detector 100 is provided to guide the inside of the detector 100 through a guide rail (not shown) or a guide groove (not shown). Accordingly, an X-ray detector 110 (hereinafter referred to as ‘first detector’) may be mounted on the table mounting portion 15 in parallel with the bottom surface.

The table mounting portion 15 on which the first detector 110 is mounted may be mounted to the table 10 to be movable in a longitudinal direction D8 of the mounting surface 11. Accordingly, all or part of the object mounted on the table 10 may be selectively captured.

As shown in FIG. 3, the subject 9 (for example, the patient) is mounted on, for example, the mounting surface 11 of the table 10, and the X-ray source 70 is positioned in the direction of the mounting surface 11 (e.g., X-rays are irradiated in a direction perpendicular to the ground). X-rays transmitted through the subject 9 are received by the first detector 110 inserted into and mounted on the table mounting portion 15.

Referring to FIGS. 1, 2 and 4, the stand 20 includes a main body 22 extending in a direction substantially perpendicular to the bottom surface, a moving groove 23 formed along at least one surface of the main body 22 along the longitudinal direction of the main body 22, a support portion 24, which has one end inserted into the moving groove 23 so as to be movable in a vertical direction d6 along the moving groove 23, and the other end of the support portion 24 is coupled to a stand mounting portion 25, which is formed so that the detector 100 can be inserted. A rotating shaft member (not shown) is provided between the support portion 24 and the stand mounting portion 25, and the stand mounting portion 25 is provided so that rotation is possible in a predetermined rotation direction d7. Accordingly, the stand mounting portion 25 may face a direction substantially perpendicular to the bottom surface (that is, a direction in which the incidence surface is horizontal to the bottom surface), and may capture the whole or a part of the subject 9. The main body 22 may be moved in the longitudinal direction d6 and/or tilted in the predetermined direction d7 so that all or a part of the subject may be photographed at various angles. According to an embodiment, the stand 20 may be formed on at least one side wall of the X-ray imaging chamber. In this case, the side wall of the X-ray imaging chamber functions as the main body 22.

The stand mounting portion 25 may be withdrawn from and/or inside the stand mounting portion 25, for example, via a guide rail (not shown), a guide groove (not shown), or the like.

As shown in FIG. 4, the subject 9 may be located in front of the stand 20, and the X-ray source 70 whose position is adjusted by the post frame 50 may be irradiated with X-rays to the subject 9. A second detector 120 mounted on the stand mounting portion 25 of the stand 20 detects the X-rays transmitted through the subject 9.

An X-ray detector 130 (hereinafter referred to as ‘third detector’) may be used for X-ray imaging without being mounted on the table 10 or the stand 20. For example, as shown in FIG. 5, the third detector 130 is not fixedly mounted to the table mounting portion 15 or the stand mounting portion 25, and is provided with the X-rays irradiated from the outside of the table 10 and the stand 20 for using in detection. In this case, the user may cause the subject 9 to hold the X-ray detector 130, or the X-ray detector 130 is mounted on a part of the subject 9 or alternatively, the third detector 130 is disposed between the back of a chair (not shown), the table 10 or a wheelchair (not shown), and the subject 9. By adjusting the position and emission direction of the X-ray source 70 to face the incident surface 101 of the third detector 130, the subject 9 can be captured at various positions, directions, and angles. Therefore, it is possible to perform custom shooting in response to the state of the subject 9. When the third detector 130 is used without being mounted on the table 10 or the stand 20, a grid may be further mounted on the third detector 130.

The workstation 400, in one embodiment, controls various components and devices within the X-ray imaging apparatus 1, such as the X-ray source 70 and the detector 100, receives electrical signals from the detector 100, and is provided to provide the user with an image corresponding to the received electrical signal.

The workstation 400 includes a main body for performing calculation and control processing of various data, and the user interface 410 connected to the main body by wire or using a wireless communication network.

The user interface 410 may include an input 411 for receiving a predetermined command from the user and a display 412 for displaying an image corresponding to the X-ray detected by the detector 100.

The input 411 may receive a command corresponding to the user's operation, for example, a driving command of the X-ray imaging apparatus 1 or a command for the emission position and the emission direction of the X-ray source 70 according to the user's operation. For example, the input 411 may receive an X-ray emission command for the table 10 or an X-ray emission command for the stand 20 by the X-ray source 70.

The input 411 may include, for example, a physical button (e.g., may include a physical keyboard), a mouse device, a trackball, a trackpad, a touch screen, a touch pad, a knob, a jog shuttle, an operation stick, a touch sensor and/or it may include a motion detection sensor, etc., if necessary, and may include a variety of interfaces connected to an external device for receiving data. Here, the interface may include, for example, data such as a universal serial bus (USB) terminal 171, a high definition multimedia interface (HDMI) terminal, a digital visual interface (DVI) terminal, a SATA terminal, or a Thunderbolt terminal, therefore at least one of the various interface terminals capable of transmitting and receiving may be included.

The display 412 may visually output data and provide the data to the user. The display 412 may be implemented using, for example, a display panel. The display panel may be, for example, a liquid crystal display (LCD) panel, a light emitting diode (LED) display panel, an organic light emitting diode (OLED) display panel, an active organic light emitting diode, or an active-matrix organic light emitting diode display panel. According to an exemplary embodiment, a touch panel capable of detecting a touch manipulation may be further formed on the display 412.

According to an embodiment, the workstation 400 may further include a sound output device (not shown) such as a speaker or an earphone.

Referring to FIGS. 6 to 15, the X-ray imaging apparatus includes a plurality of the detectors 110, 120, and 130, and the detector 110 mounted at a desired position among the plurality of detectors 110, 120, and 130. For example, the following description will be given of an embodiment using two detectors (hereinafter ‘first detector’ and ‘second detector’) for convenience of description. However, the embodiment(s) described below are not applicable only when using the two detectors 110 and 120, and obviously, it is also applicable to the case where three or more of the detectors 110, 120, 130, etc. are used in the same embodiment or through some modification.

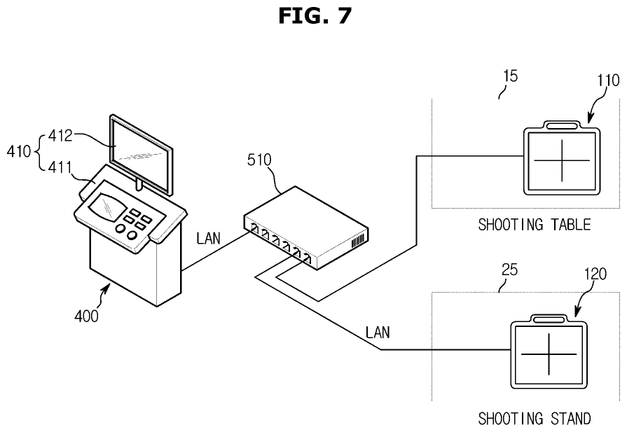

FIG. 6 is a block diagram of an embodiment of an X-ray imaging apparatus, and FIG. 7 illustrates an example of communicatively connecting an X-ray imaging apparatus and a detector.

Referring to FIG. 6, according to one embodiment, the X-ray imaging apparatus 1 may include the X-ray source 70, the first detector 110, the second detector 120, and the workstation. 400. The workstation 400 may transmit and receive mutual commands or data to at least one of the X-ray source 70, the first detector 110, and the second detector 120 through a wired/wireless communication network 500 (may be implemented using at least one of the above-described wired communication network and wireless communication network.). As described above, the X-ray source 70 may emit X-rays. When the user manipulates the input 411 to set the position and emission direction of the X-ray source 70 toward the subject 9, X-rays emitted from the X-ray source 70 are incident on the subject 9, and all or part of the incident X-rays may be absorbed or transmitted through the subject 9 by all or part of the material inside the subject 9 (e.g., bone) according to the attenuation coefficient of the material inside the subject 9, and the X-ray source 70 receives data on various parameters related to X-ray emission and control signals for preparing for or starting operation from the workstation 400 through the wired/wireless communication network 500, and based on this, the emission of a predetermined X-ray may be prepared or initiated.

The first detector 110 is provided to be carried by the user as described above, and is mounted on the table mounting portion 15 of the table 10, or mounted on the stand mounting portion 25 of the stand 20, and/or may be disposed independently of the table 10 or the stand 20. If the first detector 110 is located on a radiation path of the X-rays emitted from the X-ray source 70, the X-rays emitted from the X-ray source 70 are incident on the incident surface to the first detector 110, and the first detector 110 is incident on the incident surface. The first detector 110 may acquire an image signal corresponding to the incident X-rays. In this case, if the subject 9 is positioned between the first detector 110 and the X-ray source 70, the X-rays incident on the first detector 110 may be X-rays transmitted through the subject 9.

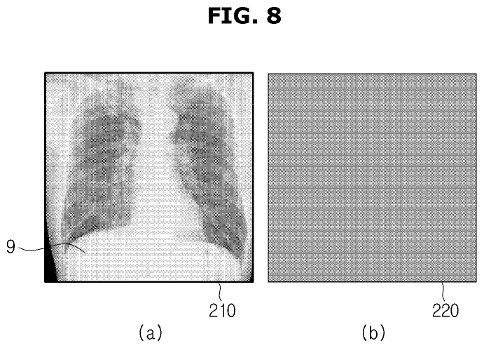

The first detector 110 may acquire an image signal even when the X-rays are hardly incident. In other words, a readout circuit 106 of the first detector 110 may perform an operation of reading an electrical signal (for example, a signal having a small size) from a light receiving element 104 even when the X-rays are not incident. In this case, since the X-rays are not incident at all or only a very small amount is incident on the first detector 110, the X-ray imaging apparatus 1 acquires an image (220 in FIG. 8) consisting only of pixels having substantially the same bright pixel values. If the subject 9 does not exist between the first detector 110 and the X-ray source 70, the image 220 may be made of only pixels having the same pixel value.

In an embodiment, the first detector 110 may include a detector controller 114, a detector communicator 115, and a detector storage 116.

The detector controller 114 may control the overall operation of the first detector 110. For example, the detector controller 114 may control an operation of reading an electrical signal corresponding to the X-ray performed by the detection panel 130. Alternatively, the operation of each component may be controlled, such as an operation of transmitting and receiving a command and/or data of the detector communicator 115 or a data storing operation of the detector storage 116. The detector controller 114 may be implemented using a predetermined processor including at least one chip in which an integrated circuit is formed.

The detector communicator 115 may transmit and/or receive various signals and data with the workstation 400 through the wired/wireless communication network 500 connected to the wired/wireless communication network 500.

For example, referring to FIG. 7, the detector communicator 115 of the first detector 110 may be connected to a network hub 510 through a cable or the like. The network hub 510 may be communicatively connected to a communicator 460 of the workstation 400 through a cable or the like. Accordingly, the detector communicator 115 and the communicator 460 of the workstation 400 may be connected to each other to enable communication. FIG. 7 illustrates an example of building the above-described communication network 500 using the network hub 510 connected by wire, but the communication network 500 is not limited thereto. For another example, the detector communicator 115 and the communicator 460 of the workstation 400 may be connected to each other via the communication network 500 established using a wireless router or the like.

For example, referring to FIG. 7, the detector communicator 115 of the first detector 110 may be connected to the network hub 510 through a cable or the like. The network hub 510 may be communicatively connected to the communicator 460 of the workstation 400 through a cable or the like. Accordingly, the detector communicator 115 and the communicator 460 of the workstation 400 may be connected to each other to enable communication. FIG. 7 illustrates an example of building the above-described communication network 500 using the network hub 510 connected by wire, but the communication network 500 is not limited thereto. For another example, the detector communicator 115 and the communicator 460 of the workstation 400 may be connected to each other via the communication network 500 established using a wireless router or the like.

In an embodiment, the detector communicator 115 transmits a readout electrical signal to the workstation 400 so that the workstation 400 displays an X-ray image corresponding to the electrical signal, and/or one of the detectors 110 and 120 may determine one of the detectors 110 or 120 estimated to have incident X-rays. In addition, the detector communicator 115 receives a control signal associated with the operation of the first detector 110 from the workstation 400, and if necessary, a signal related to the operation of the first detector 110 may be transmitted to the workstation 400. In detail, for example, the detector communicator 115 may receive a preparation command (hereinafter referred to as ‘operation preparation command’) for receiving an X-ray and reading an electrical signal from the workstation 400, and the detector controller 114 may receive the received operation preparation. The X-ray detection operation is prepared in response to the command. In addition, the detector communicator 115 may transmit a corresponding signal (hereinafter referred to as ‘response signal’) to the workstation 400 in response to the reception of the operation preparation command and/or the preparation for the X-ray detection operation. In addition, the detector communicator 115 may receive information on X-ray imaging start (i.e., X-ray imaging start information) from the workstation 400 according to an exemplary embodiment.

The detector storage 116 may temporarily or non-temporarily store data and a program for the operation of the first detector 110.

According to an embodiment, the detector storage 116 may store identification information or updated identification information (for example, may be implemented as an Internet protocol address) assigned to the first detector 110.

The detector storage 116 may include, for example, at least one of a main memory and an auxiliary memory. The main memory may be implemented using a semiconductor storage medium such as ROM and/or RAM. The ROM can include, for example, conventional ROM, EPROM, EEPROM, and/or MASK-ROM. The RAM may include, for example, DRAM and/or SRAM. Secondary storage devices include flash memory devices, SD (Secure Digital) cards, solid state drives (SSDs), hard disk drives (HDDs), magnetic drums, compact discs (CDs), and DVDs (DVDs), or at least one storage medium capable of permanently or semi-permanently storing data such as optical media such as a laser disk, a magnetic tape, a magneto-optical disk and/or a floppy disk.

The second detector 120 is provided to be physically separated from the first detector 110.

In one embodiment, the second detector 120 may include a detector controller 124, a detector communicator 125, and a detector storage 126. In some embodiments, the detector controller 124, the detector communicator 125, and the detector storage 126 of the second detector 120 may be substantially the same as the detector controller 114, the detector communicator 115, and the detector storage 116 of the first detector 110, respectively. For example, the detector communicator 125 transmits a readout electrical signal to the workstation 400, receives an operation preparation command for the second detector 120 from the workstation 400, or receives an operation preparation command, and/or transmits a response signal to the workstation 400 in response to the preparation for the X-ray detection operation, and/or receives a control signal for initiating X-ray imaging from the workstation 400. In addition, the detector communicator 125 may be communicatively connected to the communicator 460 of the workstation 400 through the network hub 510 described above. As described above, since the detector controller 114, the detector communicator 115, and the detector storage 116 of the first detector 110 have been described in detail, detailed descriptions of the same detector controller 124, the detector communicator 125, and the detector storage 126 will be omitted. Of course, the detector controller 124, the detector communicator 125, and the detector storage 126 may be provided by partially modifying the detector controller 114, the detector communicator 115, and the detector storage 116 of the first detector 110.

According to an embodiment, the workstation 400 may include the user interface 410, the controller 450, the communicator 460, and a storage 470.

The user interface 410 may receive a command from the user, and/or provide various types of information to the user visually, acoustically, and/or tactilely. The user interface 410 may include, for example, the input 411 and the display 412. Since the detailed structure and operation of the user interface 410 including the input 411 and the display 412 have already been described in detail, a detailed description thereof will be omitted.

The controller 450 may perform arithmetic processing and/or control operations necessary for the overall operation of the X-ray imaging apparatus 1. For example, the controller 450 may control the X-ray source 70 to irradiate the subject 9 with X-rays of a predetermined intensity at a predetermined dose.

The controller 450 may drive an application stored in the storage 470 to perform a predefined operation, determination, processing, and/or control operation. Here, the application may be stored in the storage 470, the program may be previously created by the designer and stored in the storage 470, or alternatively, the X-ray imaging apparatus 1 may be obtained or updated through an electronic software distribution network accessible through a wired or wireless communication network.

The controller 450 may control the X-ray source 70 by generating a control signal related to the operation of the X-ray source 70 and/or various parameters related to X-ray emission.

In addition, the controller 450 generates an operation preparation command for the first detector 110 and the second detector 120, and determines whether a response signal corresponding to the operation preparation command is transmitted from the first detector 110 and the second detector 120, and based on the determination result, determines whether the first detector 110 and the second detector 120 are normally connected, whether the first detector 110 and the second detector 120 perform normally, and/or whether they are ready to receive X-rays. Accordingly, the operation of the X-ray source 70 and the operations of the first detector 110 and the second detector 120 may be synchronized with each other.

The transmission of the control signal for at least one of the above-described X-ray source 70, the first detector 110, and the second detector 120 may be performed in response to the user's input of an X-ray emission command through the input 411.

In addition, the controller 450 may acquire an X-ray image based on an electrical signal transmitted from each of the first detector 110 and the second detector 120, and the user can control the display 412 of the user interface 410 to obtain the X-ray image. In detail, for example, the controller 450 may generate an X-ray image of a form that can be viewed by a human based on an electrical signal output from the detection panel 130 of each of the first detector 110 and the second detector 120. The image generated by the controller 450 may include a still image and a moving image, where the moving image may be implemented by the display 412 continuously outputting two or more still images.

If necessary, the controller 450 may further perform various image processing on the generated X-ray image. For example, the controller 450 may add a sharpness effect to all or part of the X-ray image generated by using a high pass filter, or may use a low pass filter. Blur effects may be added to all or part of the image. As another example, the controller 450 may generate a stereoscopic image based on the generated plurality of X-ray images, or may add color to the generated X-ray image as previously defined. In addition, the controller 450 may further perform various image processing according to the user's selection.

In this case, the controller 450 may determine one of the detectors 110 and 120, which are estimated to have incident X-rays, from among the detectors 110 and 120 through various methods. This will be described later.

The controller 450 may be, for example, a central processing unit (CPU), a micro controller unit (MCU), a microcomputer (Micom), an application processor (AP), or an electronic control unit (ECU) and/or other electronic devices capable of generating various calculations and control signals. These devices may be implemented, for example, using one or more semiconductor chips and associated components.

In addition, the controller 450 may be implemented by a semiconductor chip and related components embedded in the main body of the workstation 400 described above, and/or the X-ray source 10. It may also be implemented by a semiconductor chip and associated components provided in the auxiliary user interface device 80 mounted in the X-ray source 10.

The communicator 460 is connected to the wired/wireless communication network 500 so that the workstation 400 communicates with the X-ray source 70, the detector communicator 115 of the first detector 110 and/or the detector communicator 125 of the second detector 120. For example, the communicator 460 may transmit various control signals generated by the controller 450 (for example, an operation preparation command, an X-ray imaging start command, or information about the same) to the X-ray source 70, the first detector 110, and/or to the second detector 120, or receives a response signal from the first detector 110 and/or the second detector 120, and/or receives electrical signals from each of the first detector 110 and the second detector 120.

The storage 470 may store various information related to the operation of the X-ray imaging apparatus 1 or may temporarily or non-temporarily store an image acquired by the controller 450. According to an embodiment, the storage 470 may temporarily or non-temporarily store an image transmitted from the detectors 110 or 120.

In addition, the storage 470 may store, for example, the history of each of the detectors 110 and 120. For example, the storage 470 may store the history of the locations where the detectors 110 and 120 were previously mounted. For example, the storage 470 may store statistical information about the mounting positions of the detectors 110 and 120, information about the mounting position immediately before the detectors 110 and 120, or various information related to the use of the detectors 110 and 120. More specifically, for example, the storage 470 stores information such as, the first detector 110 being mounted to the table mounting portion 15 of the table 10 mainly or immediately before, and the second detector 120 being mounted on the stand mounting portion 25 of the stand 20 mainly or immediately before.

In addition, the storage 470 may store location recognition results for each of the detectors 110 and 120 according to the operation of the controller 450.

The storage 470 may be implemented using at least one of a magnetic disk storage device, a magnetic tape storage device, and a semiconductor storage device, according to an embodiment. Here, the semiconductor storage device may be implemented by using various media that can store data.

FIG. 6 illustrates an example in which the storage 470 is provided inside the main body 310 of the workstation 400, but this is merely an example, and an install location of the storage 470 is not limited thereto. For example, the storage 470 may be implemented using a memory device mounted on a board embedded in the device(s) installed inside a radiography room. More specifically, for example, the storage 470 may include the moving carriage 40 provided for transporting the X-ray source 70, the auxiliary user interface device 80 mounted on the X-ray source 70, the table 10, and the stand 20. When the storage 470 is provided in the device(s) installed in the radiography room as described above, the history of each of the detectors 110 and 120 and/or the position recognition result of each of the detectors 110 and 120 may be used, or may store a variety of information other than these. In addition, the storage 470 may be implemented using a separate storage device provided outside the workstation 400. In this case, the storage device provided separately from the workstation 400 may be communicatively connected to the workstation 400 through a cable or a wireless communication network.

Hereinafter, an example in which the controller 450 determines one of the plurality of detectors 110 and 120 to determine which of the detectors 110 or 120 is estimated to have incident X-rays will be described.

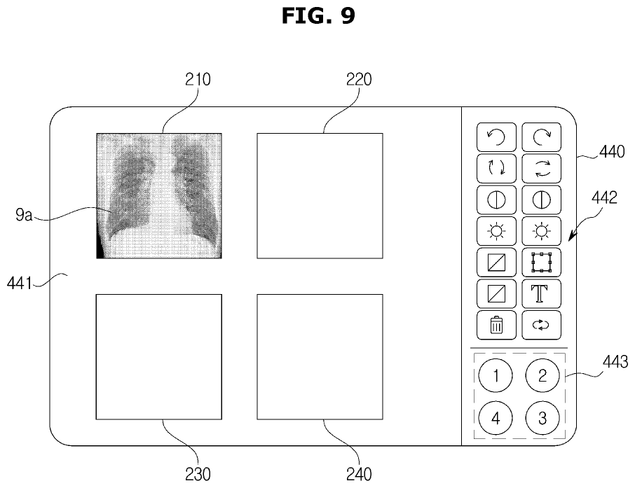

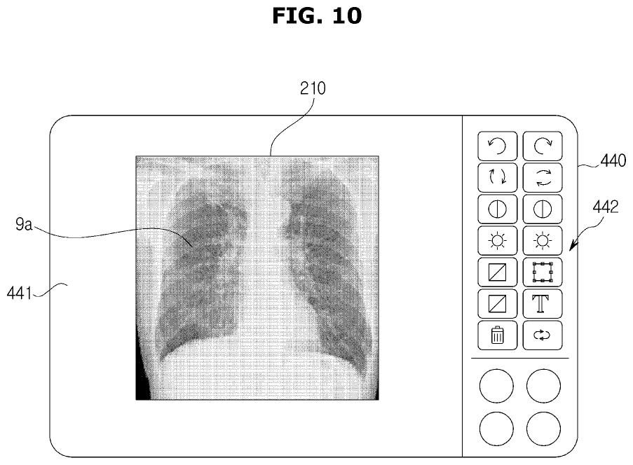

FIG. 8 is a diagram illustrating an example of an image acquired from each detector, FIG. 9 is a diagram illustrating an example of a screen on which a plurality of images acquired from each detector is displayed, and FIG. 10 is a diagram illustrating an example of a screen on which one selected image is displayed.

As shown in FIG. 2, the first detector 110 is mounted to the table mounting portion 15 of the table 10 by the user, and the second detector 120 is mounted to the stand mounting portion 25 of the stand 20. Each of the first detector 110 and the second detector 120 is connected to the workstation 400 through, for example, the network hub 510.

As illustrated in FIG. 3, the subject 9 may be mounted on the mounting surface 11 of the table 10, and the user (radiographer, etc.) may input information about a shooting position through the input 411. In other words, the user may input information indicating that the subject 9 is positioned on the table 10. Accordingly, the X-ray imaging apparatus 1 prepares and starts X-ray imaging of the input imaging position, that is, the table 10.

According to the information on a capturing position, the X-ray source 70 is appropriately moved around the table 10 by moving and/or extending at least one of the above-described guide rail 32, the moving carriage 40, and the post frame 50. According to the operation of the rotary joint 60, its emission direction is controlled to face the first detector 110 mounted on the table mounting portion 15.

Before, after, or at the same time, the workstation 400 transmits an operation preparation command to each of the first detector 110 and the second detector 120, and each of the first detector 110 and the second detector 120 prepares for the X-ray detection operation in response to the reception of the operation preparation command. Each of the first detector 110 and the second detector 120 sequentially transmits a response signal corresponding to the operation preparation command to the workstation 400. The workstation 400 determines that the first detector 110 and the second detector 120 are ready according to the response signals transmitted from the first detector 110 and the second detector 120, respectively.

In turn, the workstation 400 transmits an X-ray emission command to the X-ray source 70, and the X-ray source 70 emits X-rays in response. In this case, the workstation 400 notifies that X-ray imaging has started, as necessary. Alternatively, the X-ray imaging start information (or command) for giving an instruction related thereto may be further transmitted to each of the first detector 110 and the second detector 120.

When X-rays are emitted from the X-ray source 70, each of the first detector 110 and the second detector 120 operates independently to readout an electrical signal from the light receiving element 104. The readout signal, that is, the electrical signal regarding a first image 210 and the electrical signal regarding the second image 220 are transmitted to the workstation 400.

Due to the position and emission direction of the X-ray source 70 as described above, the first detector 110 mounted on the table mounting portion 15 receives the X-ray, while the second detector 120 mounted on the stand mounting portion 25 receives little or no X-rays.

Accordingly, in the first image 210 corresponding to the electrical signal read by the first detector 110, as shown in FIG. 8A, the subject 9 through which the X-rays are transmitted appears, whereas the X-rays are partially or not at all incident to the second detector 120. As shown in FIG. 8B, the subject 9 does not appear at all in the second image 220 corresponding to the electrical signal read by the second detector 120.

The controller 450, in one embodiment, controls the display 412 to display a predetermined graphical user interface 440. In this case, as illustrated in FIG. 9, the controller 450 displays the images 210 and 220 corresponding to each of the plurality of detectors 110 and 120 in one region 441 of the graphical user interface 440. According to an exemplary embodiment, the images 210 and 220 corresponding to each of the plurality of detectors 110 and 120 may be sequentially displayed. If a signal for an image is transmitted from each of three or more of the detectors, the controller 450 may control the display 412 to further display images 230 and 240 corresponding to each signal simultaneously or sequentially in the one region 441.

The user may manipulate the input 411 to select the most appropriate image (i.e., an image detected by a detector disposed at a desired location of the user, and may include a detector determined to have incident X-rays) among the plurality of images 210 to 240 displayed in the one region 441 of the graphic user interface 440. For example, the user may select the first image 210 in which all or a portion 9a of the subject is displayed from among the plurality of images 210 to 240. The controller 450 may determine that the detector corresponding to the first image 210, that is, the first detector 110 mounted on the table mounting portion 15 is a detector disposed at a desired position of the user. According to an exemplary embodiment, a guide image 443, such as an icon, may be displayed in another region 442 of the graphical user interface 440 for convenience of the user's selection.

The controller 450 determines and recognizes the selected image, for example, a detector corresponding to the first image 210, for example, the first detector 110, as a capturing position, for example, a detector corresponding to the table 10. Decision and recognition results can be stored in the storage 470. Therefore, in the case where another capturing position using the table 10 as the capturing position is further performed after the capturing is finished, the controller 450 drives only the recognized detector, for example, the first detector 110, or alternatively, as described below, the first image 210 obtained by the first detector 110 is given priority to display the display 412 in advance of the second image 220 obtained by the second detector 120.

In addition, the controller 450 may control the display 412 such that only the selected first image 210 is displayed on the graphical user interface 440. Accordingly, the second image 220 acquired by the other detector 120 is not displayed on the display 412. If necessary, the controller 450 may erase the second image 220 acquired by the other detector 120 from the storage 470, for example, the main memory device.

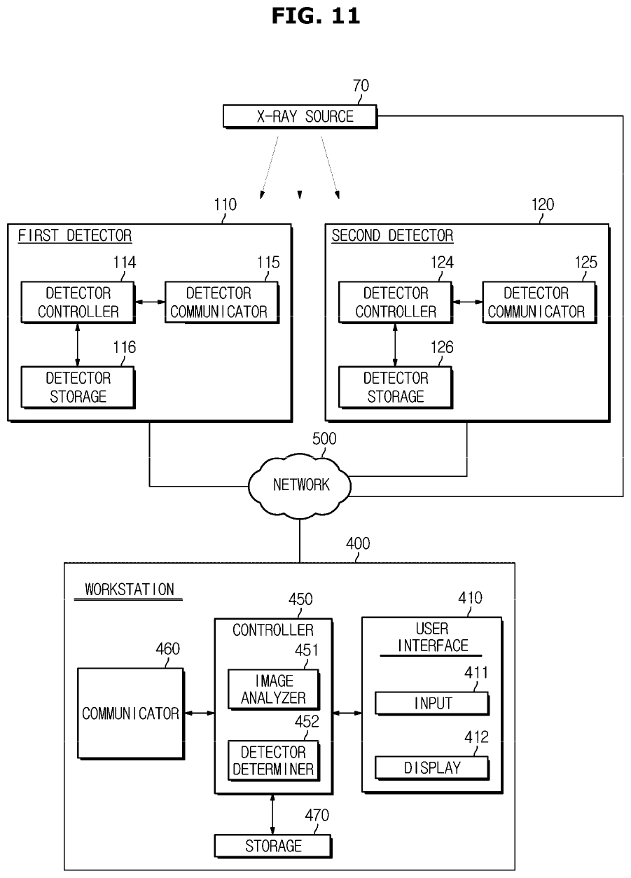

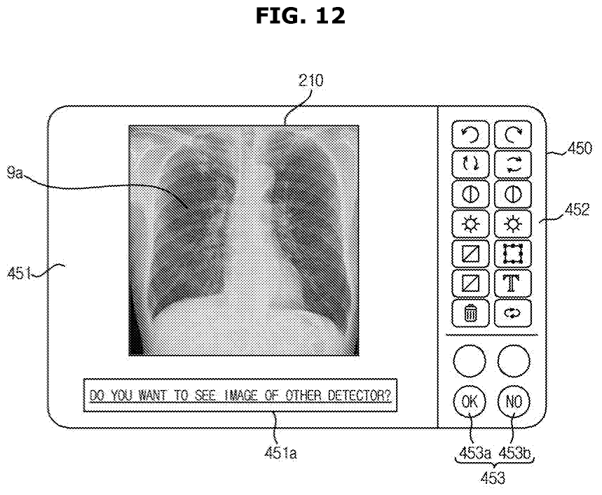

FIG. 11 is a block diagram of another embodiment of an X-ray imaging apparatus, and FIG. 12 is a diagram illustrating an example of a screen on which an automatically selected image is displayed.

Referring to FIG. 11, the X-ray imaging apparatus 1 according to another embodiment may include the X-ray source 70, the first detector 110, the second detector 120, and the workstation 400. The workstation 400 may transmit and receive mutual commands or data to at least one of the X-ray source 70, the first detector 110, and the second detector 120 through the predetermined wired/wireless communication network 500.

The X-ray source 70 is designed to emit X-rays.

The first detector 110 and the second detector 120 are physically separated from each other. According to an embodiment, the first detector 110 includes the detector controller 114, the detector communicator 115 and the detector storage 116, and the second detector 120 may include the detector controller 124, the detector communicator 125, and the detector storage 126.

In one embodiment, the workstation 400 may include the user interface 410 including the input 411, the display 412, the controller 450, the communicator 460, and the storage 470.

The detailed structures and operations of the X-ray source 70, the first detector 110, the second detector 120, the user interface 410, the communicator 460, and the storage 470 described above will be omitted.

As shown in FIG. 11, the controller 450 may include an image analyzer 451 and a detector determiner 452. The image analyzer 451 and the detector determiner 452 may be logically separated from each other or may be physically separated from each other.

The image analyzer 451 may compare the plurality of images 210 and 220 to determine the image corresponding to the detector 110 or 120 from which the X-rays are estimated to be incident among the plurality of detectors 110 and 120. Specifically, the image analyzer 451 is an image in which the subject 9 actually exists, that is, the image 210 or 220 obtained from the detector 110 or 120 that receives the X-rays transmitted through the subject 9 may be determined.

For example, when the image analyzer 451 receives the first image 210 from the first detector 110 and the second image 220 from the second detector 120, an image in which the subject 9 is present may be determined from the first image 210 and the second image 220 by analyzing the first image 210 and the second image 220 or comparing the first image 210 and the second image 220 with each other.

More specifically, the image analyzer 451 acquires pixel values of each pixel included in the first image 210, for example, an RGB value or a contrast value, and it is determined whether the pixel value of each pixel exceeds a predefined value, and/or determined whether or not the pixel values of the respective pixels are significantly different from each other, to estimate whether the subject 9 exists in the first image 210. For example, if a fairly large area in the first image 210 is bright and other portions are dark, the image analyzer 451 determines that the internal structure of the subject 9, such as a bone, appears in the bright portion, and determines the displayed first image includes the subject 9. More specifically, for example, the RGB value of a fairly large area in the first image 210 is approximately (FFFFFF) or approximate to this, and the RGB values of the other zones are approximately (000000) or close thereto, then it may be determined that the internal structure of the subject 9 appears in a portion corresponding to approximately (FFFFFF). The image analyzer 451 may determine whether the second image 220 includes the subject 9 through the same process as described above with respect to the second image 220. Accordingly, the image analyzer 451 may determine the image 210 or 220 in which the subject 9 is displayed among the first image 210 and the second image 220.

In addition, the image analyzer 451 may directly compare the first image 210 and the second image 220 to determine the image 210 or 220 having more relatively bright portions as an image in which the subject 9 appears.

In response to the analysis result, the controller 450 controls the display 412 to control the display 412 to display the graphical user interface 440 as shown in FIG. 12. A predetermined image, for example, the first image 210 according to the analysis result of the image analyzer 451 may be displayed in the one region 441 of the graphical user interface 440. In other words, the first image 210 that is determined to include the portion 9a may be displayed in the one region 441.

In addition, the controller 450 may receive a confirmation (confirmation or response) as to whether the displayed first image 210 is an appropriate image from the user. To this end, the controller 450 may cause the display 412 to display a message 451a for requesting approval or to further display a guide image 453 for assisting the user in inputting an approval command. In this case, the message 451a may include a query for confirming whether the displayed first image 210 is an appropriate image or a query for confirming whether to view the second image 220 other than the displayed first image 210. The message 451a may be displayed at any position of the graphical user interface 440 and may be displayed in the form of a pop-up window according to an embodiment. The guide image 453 may be displayed, for example, in a region 452 of the graphical user interface 440 and may include an image 453a associated with approval and an image 453b associated with rejection. According to the user's manipulation corresponding to either of the two images 453a and 453b, the user's selection as to whether the displayed first image 210 is an appropriate image may be input to the workstation 400.

If the user inputs an approval command that the displayed first image 210 is an appropriate image, the controller 450 continuously displays the first image 210 in response. On the contrary, if the user inputs a disapproval command indicating that the displayed first image 210 is not an appropriate image, the controller 450 responds to display another image different from the first image 210, for example, the second image 220. The display may be controlled to be displayed in the same or partially modified manner as the first image 210. After the second image 220 is displayed, the controller 450 displays the message 451a for requesting approval by the display 412 as described above. Alternatively, the guide image 453 may be further displayed to assist the user in inputting an approval command. According to the user's approval or disapproval command, it may be determined whether the second image 220 is an appropriate image.

If the user inputs an approval command that the displayed first image 210 or the second image 220 is an appropriate image, the detector determiner 452 of the controller 450 determines and recognizes the detector corresponding to the detector corresponding to the first image 210, that is, the detector corresponding to the first detector 110 or the second image 220, that is, the shooting position where the second detector 120 is desired, that is, the detector mounted on the table 10, and stores decision and recognition results in the storage 470. Accordingly, the detectors 110 and 120 corresponding to the photographing positions desired by the user can be determined.

In addition, descriptions of other operations or structures of the controller 450, which are redundant with those described above, will be omitted.

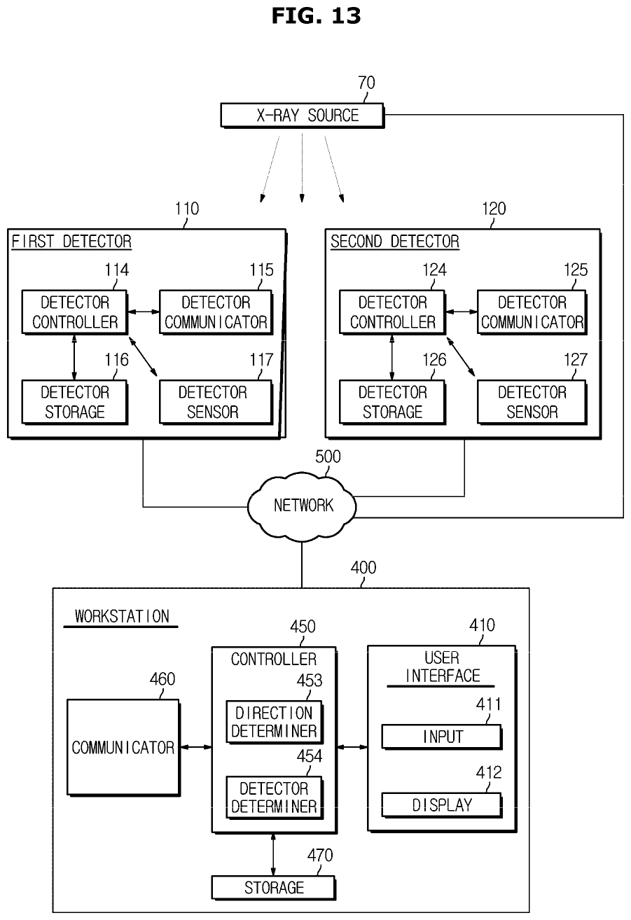

FIG. 13 is a block diagram of another embodiment of an X-ray imaging apparatus. Referring to FIG. 13, according to another embodiment, the X-ray imaging apparatus 1 may include the X-ray source 70, the first detector 110, the second detector 120, and the workstation. 400. The workstation 400 may transmit and receive mutual commands or data to at least one of the X-ray source 70, the first detector 110, and the second detector 120 through the predetermined wired/wireless communication network 500.

The X-ray source 70 is designed to emit X-rays.

The first detector 110 and the second detector 120 are physically separated from each other. According to an embodiment, the first detector 110 may include the detector controller 114, the detector communicator 115, and the detector storage 116, and may further include a direction sensor 117. In addition, the second detector 120 may also include the detector controller 124, the detector communicator 125, the detector storage 126, and a direction sensor 127.

The direction sensors 117 and 127 detect a direction that the first detector 110 and the second detector 120 face. Here, the direction toward which the first detector 110 and the second detector 120 may include a direction in which X-rays are normally incident during X-ray imaging, for example, the normal direction of the incident surface 101 may be included.

The direction sensors 117 and 127 may be provided on the back side or the side of the X-ray detector 130, or may be provided inside the X-ray detector 130. As long as it does not affect the detection of X-rays, the direction sensors 117 and 127 can be installed at arbitrary positions.

The direction sensors 117 and 127 may include, for example, a magnetic sensor and/or a tilt sensor. However, the direction sensors 117 and 127 are not limited thereto, and may include other types of sensors capable of measuring or detecting a direction in which the incident surface 101 of the X-ray detector 100 faces.

The magnetic sensor refers to a sensor that detects the presence, direction, strength, and the like of a magnetic field. The magnetic sensor may include a linear magnetic sensor and a nonlinear magnetic sensor. The linear magnetic sensor refers to, for example, a magnetic sensor that linearly outputs a value corresponding to the strength of a magnetic field, such as a Hall sensor. The nonlinear magnetic sensor refers to, for example, a magnetic sensor that outputs an on/off signal depending on whether the intensity of the magnetic field is greater than or equal to a threshold, such as a Hall Integrated Circuit (IC).

The magnetic sensor senses the direction of the different magnetic fields depending on whether the X-ray detectors 110 and 120 are mounted on the table mounting portion 15, the stand mounting portion 25, or used independently, and detects the direction of the electric field to output the signal corresponding to the detection result. The electrical signal may be transmitted to the controller 450 through the communicator 460 of the detector communicators 115 and 125, the communication network 500, and the workstation 400.

The tilt sensor is a sensor that detects the degree of inclination of an object with respect to the direction of gravity. For example, it may include an acceleration sensor that detects an inclination by measuring a degree parallel to gravity acceleration, or a gyro sensor that detects an inclination by measuring a rotation direction or rotation angle according to a movement.

The direction sensors 117 and 127 may detect and measure the degree of inclination of the X-ray detectors 110 and 120, and output electrical signals corresponding to the detection and measurement results. For example, the direction sensors 117 and 127 may output a signal corresponding to the first detector 110 that is directed in a direction substantially perpendicular to the bottom surface, or the second detector 120 may output a signal indicating that the normal direction of the incident surface is oriented in a direction substantially horizontal to the bottom surface. The output electrical signal may be transmitted to the controller 450 through the communicator 460 of the detector communicators 115 and 125, the communication network 500, and the workstation 400.

The detector controllers 114 and 124 may cause the direction sensors 117 and 127 to start operation and/or sense and/or detect from the direction sensors 117 and 127 based on a control signal transmitted from the workstation 400. Alternatively, the measurement results may be collected, and the sensing and/or measurement results may be controlled to be transmitted to the workstation 400.

The controller 450 of the workstation 400 may, in one embodiment, include a direction determiner 453 and a detector determiner 454. The direction determiner 453 and the detector determiner 454 may be logically separated from each other, or may be physically separated from each other.

The direction determiner 453 may determine the predetermined detectors 110 and 120 disposed at the photographing position desired by the user based on the degree of inclination (tilt) of each of the first detector 110 and the second detector 120. In other words, the direction determiner 453 determines whether the respective detectors 110 and 120 are mounted or used independently according to the direction in which the incidence planes of the first detector 110 and the second detector 120 face each other.

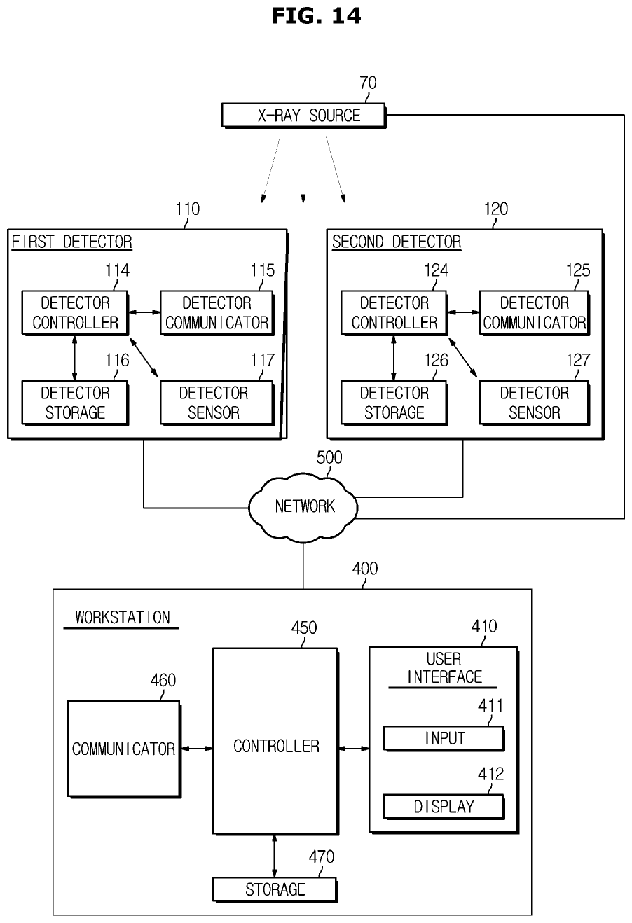

Specifically, for example, as shown in FIG. 14, if the subject 9 is positioned on the mounting surface 11 of the table 10, and the appropriately moved X-ray source 70 irradiates X-rays to the subject 9, the first detector 110 mounted on the table mounting portion 15 of the table 10 detects the X-rays passing through the subject 9. As described above, since the table mounting portion 15 is substantially horizontal with respect to the bottom surface, the first detector 110 also faces a direction substantially horizontal with respect to the bottom surface. Therefore, if the inclination of the first detector 110 is approximately horizontal with the bottom surface (e.g., if the inclination is determined to be approximately zero or close to zero), the first detector 110 is mounted on the table mounting portion 15.

For another example, as shown in FIG. 4, if the subject 9 is located in front of the stand 20 and the X-ray source 70 whose position is adjusted by the post frame 50 or the like irradiates X-rays to the subject 9, the first detector 110 mounted on the stand mounting portion 25 of the stand 20 detects the X-rays transmitted through the subject 9. As described above, since the stand mounting portion 25 faces a direction perpendicular to the bottom surface, the first detector 110 also faces a direction perpendicular to the bottom surface. Therefore, when it is determined that the inclination of the first detector 110 is approximately perpendicular to the bottom surface (that is, when the inclination is determined to be approximately 90 degrees or approximate thereto), the first detector 110 may be used. It can be determined that it is mounted on the stand mounting portion 25.

For another example, as shown in FIG. 5, when the first detector 110 is used without being mounted to the table mounting portion 15 or the stand mounting portion 25, the detector, for example, the second detector 120 may be inclined at a predetermined angle θ with a normal direction of the bottom surface. Here, the predetermined angle θ may include another angle that is not close to 0 degrees or 90 degrees, but may also include 0 degrees or 90 degrees as necessary. Therefore, if the inclination of the second detector 120 has another angle that is significantly different from approximately 0 degrees or 90 degrees, it may be determined that the second detector 120 is used separately.

As described above, the direction determiner 453 receives a signal corresponding to the inclination of each of the detectors 110 and 120 transmitted from each of the detectors 110 and 120, and receives the signals of the respective detectors 110 and 120. Based on the slope, it is possible to determine whether each of the detectors 110 and 120 is mounted on the table mounting portion 15 or the stand mounting portion 25 or is used independently.

On the other hand, the user can operate the input 411 at the start of X-ray imaging to input a command regarding the capturing position to the workstation 400. The workstation 400 may adjust the emission position and/or direction of the X-ray source 70 based on this. The detector determiner 454 may detect the image 210 or 220 corresponding to the detector 110 or 120 or the detector 110 or 120 that is determined to have received X-rays among the plurality of detectors 110 and 120 based on the input position. More specifically, the detector determiner 454 determines when the inclination of each of the plurality of detectors 110 and 120 is determined, and correspondingly, the position of each of the plurality of detectors 110 and 120 is determined, compares this with the capturing position, among the detectors 110 and 120, and determines the detector 110 or 120 whose position corresponds to the capturing position, and determines the determined detector 110 or 120 as a detector estimated to have incident X-rays.

When the detector 110 or 120 is estimated to have incident X-rays, under the control of the controller 450, the display 412 displays the predetermined graphical user interface 440 as shown in FIG. 12. And the image 210 or 220 corresponding to the estimated detector 110 or 120 is displayed. In other words, in the one region 441 of the graphical user interface 440, the image 210 or 220 that is determined to include the portion 9a may be displayed based on the inclination of the detector 110 or 120.

As described above, the display 412 may display the message 451a for requesting approval or further display the guide image 453 for assisting the user in inputting an approval command, and the controller 450 may display the user's input. Depending on the approval, the display 412 may maintain the displayed image 210 or 220, or may control to display another image. Even after the display 412 displays another image, the controller 450 may receive the user's approval through the input 411.

As described above, the display 412 may display the message 451a for requesting approval or further display the guide image 453 for assisting the user in inputting an approval command. The controller 450 may control to display the image 210 or 220 on which the display 412 is displayed or display another image according to whether the user approves. Even after the display 412 displays another image, the controller 450 may receive the user's approval through the input 411.

Also in the embodiment described above, the controller 450 generates an operation preparation command for the first detector 110 and the second detector 120, determines whether the first detector 110 and the second detector 120 are normally connected based on whether the response signals transmitted from the first detector 110 and the second detector 120 are received, or whether they perform their normal operations and/or whether they are ready to receive X-rays.

Although the above-described method of determining the detectors 110 and 120 in which X-rays are estimated to be incident by using the inclination of the detectors 110 and 120 has been described, the controller 450 may have different values than the inclination of the detectors 110 and 120 as necessary. The detectors 110 and 120 estimated to have incident X-rays may be determined using the information.

For example, the controller 450 may determine the detectors 110 and 120 estimated to have incident X-rays based on the history of the detectors 110 and 120 stored in the storage 470. More specifically, for example, the controller 450 may determine the detectors 110 and 120 from which X-rays are estimated to be incident on the basis of the information on the positions where the specific detectors 110 and 120 are mainly mounted. For another example, the detectors 110 and 120 may determine the detectors 110 and 120 estimated to be incident on the X-rays based on the information about the position of the specific detectors 110 and 120 that were mounted in the previous capturing process.

In addition, descriptions of other operations and structures of the X-ray source 70, the first detector 110, the second detector 120, the user interface 410, the communicator 460, the controller 450, and the storage 470, which are redundant with those described above, are omitted.

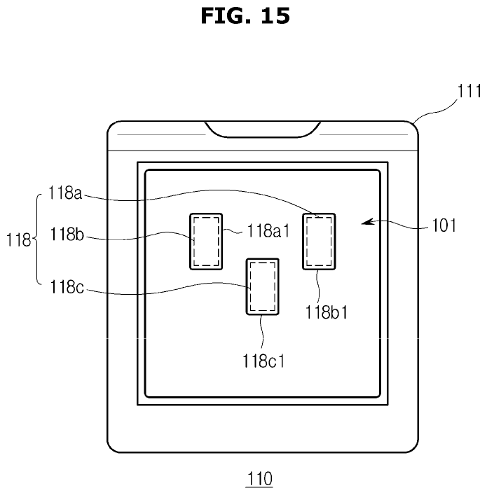

FIG. 14 is a block diagram of another embodiment of an X-ray imaging apparatus, and FIG. 15 is a diagram illustrating another embodiment of a detector.

Referring to FIG. 14, the X-ray imaging apparatus 1 according to another embodiment may include the X-ray source 70, the first detector 110, the second detector 120, and the workstation 400. The workstation 400 may transmit and receive mutual commands or data to at least one of the X-ray source 70, the first detector 110, and the second detector 120 through the predetermined wired/wireless communication network 500.

The X-ray source 70 is designed to emit X-rays.

The first detector 110 and the second detector 120 are physically separated from each other, and according to an embodiment, the first detector 110 includes the detector controller 114, the detector communicator 115, and the detector storage 116. In addition, a detector sensor 118 may be further included. Similarly, the second detector 120 may include the detector controller 124, the detector communicator 125, the detector storage 126, and a detector sensor 128.

In one embodiment, the workstation 400 may include the user interface 410 including the input 411, the display 412, the controller 450, the communicator 460, and the storage 470.

According to an embodiment, the detector sensors 118 and 128 automatically detect the exposure of the X-rays, and allow the detectors 110 and 120 to perform the X-ray light receiving operation according to the detection result.

The detector sensors 118 and 128 detect the dose of X-rays, and output an electrical signal according to the detection result to the detector controller 114. The detector controller 114 detects the X-ray dose and a predefined threshold value, and in comparison, when the dose of the X-rays is greater than the threshold, the detectors 110 and 120 may automatically perform the light receiving, reading, and outputting operations of the X-rays. Accordingly, the detectors 110 and 120 may perform an auto exposure detection (AED) operation.

For example, the detector sensor 118 may be provided between the light receiving element 104 or a semi-scattered grid (not shown) and the incident surface 101.

The detector sensor 118 may include a plurality of X-ray detectors, for example, the first X-ray detectors to three detector sensors 118a to 118c. Each of the detector sensors 118a to 118c independently detects the dose of X-rays and outputs a detection result.

Referring to FIG. 15, each of the detector sensors 118a to 118c may be installed at least at one point of the incident surface 101, respectively. For example, two of the detector sensors 118a and 118b are installed at the upper end of the incident surface 101. One of the detector sensors 118c may be installed at the lower ends of the two detector sensors 118a and 118b. According to an exemplary embodiment, markers 118a1, 118b1, and 118c1 may be further displayed on the incident surface 101 to indicate the positions of the detector sensors 118a to 118c.

As described above, when the detector sensors 118 and 128 are provided, the detectors 110 and 120 properly receive X-rays irradiated from the X-ray source 70 without transmitting an operation preparation command to the detectors 110 and 120. Therefore, synchronization of the operation of the X-ray source 70 and the operation of the respective detectors 110 and 120 becomes unnecessary. In addition, even when communication between the detectors 110 and 120 and the X-ray source 70 is impossible, the detectors 110 and 120 may receive X-rays at an appropriate point in time.

Meanwhile, the detector sensors 118 and 128 of the detectors 110 and 120 may detect the X-rays emitted due to scattering or reflection even when the X-rays emitted from the X-ray source 70 are not directly incident. That is, X-rays above the threshold required for detection may be incident on the detector sensors 118 and 128 according to circumstances. Accordingly, some detectors among the plurality of detectors 110 and 120 in the X-ray examination room perform detection of some X-rays even though the X-rays do not penetrate through the subject.

The detectors 110 and 120 provided with the detector sensors 118 and 128 may also transmit the images 210 and 220 to the workstation 400 as described above. In this case, not only the detector 110 or 120 which has performed the automatic exposure detection operation, but also the detector which has not performed the automatic exposure detection operation may output a predetermined signal corresponding to the image.

The workstation 400 displays at least two or more of each of the received images 210 and 220 so that the user selects the appropriate image 210 or 220, or analyzes the received images 210 and 220, respectively, to determine the image 210 or 220 where the subject 9 exists, and/or determines the detector 110 or 120 corresponding to an input capturing position based on the inclination of the detectors 110 and 120, and determines the image 210 or 220 corresponding to the determined detector 110 or 120. Accordingly, the workstation 400 may determine the detector 110 or 120 and/or the image 210 or 220 corresponding to the incident X-rays. The display 412 of the workstation 400 may sequentially display the determined image 210 or 220, and receive an approval or disapproval command from the user to determine whether to maintain the display of the determined image 210 or 220. In addition, the detector 110 or 120 corresponding to the estimated image 210 or 220 may be determined as the detector currently being used in response to the user's approval.

In addition, descriptions of other operations and structures of the X-ray source 70, the first detector 110, the second detector 120, the user interface 410, the communicator 460, the controller 450, and the storage 470, which are redundant with those described above, are omitted.

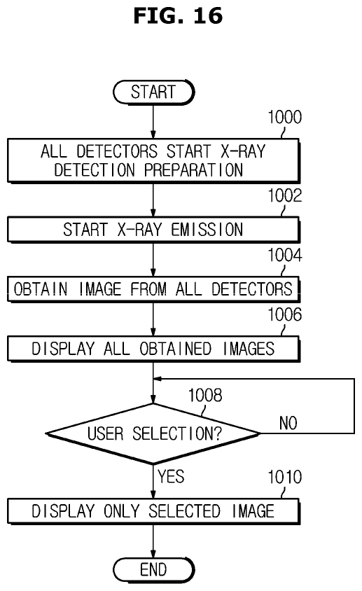

Hereinafter, various embodiments of a method for determining a position of a detector will be described with reference to FIGS. 16 to 20.

FIG. 16 is a flowchart illustrating an embodiment of a method for position determining of a detector.

As illustrated in FIG. 16, a plurality of detectors (all detectors) provided in an X-ray detecting apparatus prepare for detection of X-rays under control of a workstation or an X-ray source (1000).

The X-ray source sequentially starts to emit X-rays, and the emitted X-rays are incident on one of the detectors (1002). In this case, the X-rays incident on any one detector may include X-rays passing through a subject.

Each of the plurality of detectors reads an electrical signal. In this case, one detector reads a signal corresponding to the incident X-ray, but another detector reads a signal that is independent of or hardly related to the X-rays because the X-rays are not incident. Accordingly, a plurality of images may be obtained from each of the plurality of detectors (1004).

The workstation may display all of the plurality of images acquired by using a display provided in the workstation (1006).

A user may sequentially select one of the plurality of images. In this case, the user may check all displayed images and select an image (i.e., an image on which a subject appears) corresponding to the incident of X-rays from the identified images.

If the user selects any one of the plurality of images (Yes in 1008), the selected image is determined to be an image obtained by a detector that is estimated or determined to have incident X-rays, and a display unit of the workstation may display only the image selected by the user but no other images (1010). In addition, the workstation may recognize a detector corresponding to the selected image (i.e., a detector estimated or determined to have incident X-rays) as a detector mounted at a position where the X-rays are emitted, and store the recognition result. The stored recognition result can be used in a later X-ray emission process.

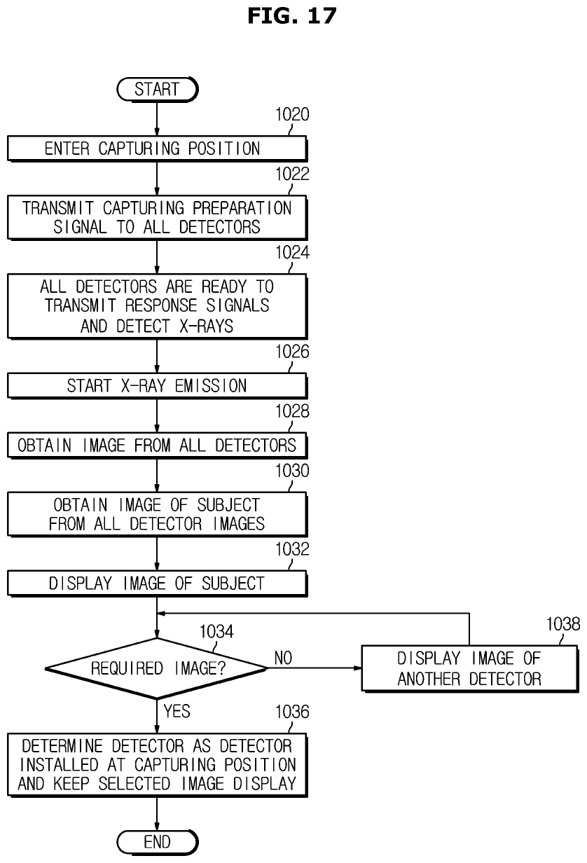

FIG. 17 is a flowchart illustrating another embodiment of a method for position determining of a detector.

Referring to FIG. 17, first, a user may input a photographing position by operating an input unit provided at a workstation of an X-ray imaging apparatus (1020). In this case, a capturing position may be a stand or a table, for example. In addition, the photographing position may be other positions than the stand and the table.

When the capturing position is input, the X-ray source of the X-ray imaging apparatus is adjusted to an emission position and/or emission direction according to the input capturing position, and a capturing preparation signal is transmitted from the workstation to a plurality of detectors (all detectors) of the X-ray imaging apparatus (1022).

The plurality of detectors receives a capturing preparation signal and enters a preparation state for detecting X-rays in response to a photographing preparation signal (1024). In addition, the plurality of detectors may transmit a response signal corresponding to the imaging preparation signal to the workstation.

When the workstation receives the response signal from the plurality of detectors, it determines that all of the detectors are ready to receive and detect X-rays. In response to the determination, the control signal may be transmitted to the X-ray source simultaneously or after a predetermined time. Accordingly, the X-ray source starts X-ray emission (1026). In this case, the workstation may transmit a control signal to the X-ray source even when a response signal is not transmitted from some of the detectors according to a predefined condition or the user's selection.

In response to the X-ray source starting the X-ray emission operation, the plurality of detectors respectively performs an operation of reading an electrical signal (1028). Accordingly, a plurality of images can be obtained. In this case, one image among the plurality of images may be an image acquired in response to light reception of the irradiated X-ray, but another image may be an image obtained without the light reception of the X-ray.

The workstation receives an electrical signal corresponding to the plurality of images, obtains the plurality of images corresponding to each of the plurality of detectors, and analyzes or compares each of the plurality of images corresponding to the plurality of detectors, and among the plurality of images. An image including a subject is detected and acquired (1030). For example, the workstation may acquire an image including a subject from among the plurality of images by using pixel values (e.g., RGB values) of pixels of each image. In this case, the image including the subject corresponds to a detector estimated to have incident X-rays.

The workstation displays the acquired image on a display and provides the acquired image to the user (1032).

The user determines whether the acquired image is an intended image (i.e., an image of an internal structure of the subject) in response to the display of the acquired image (1034).

If the displayed image is the image requested by the user (YES in 1034), the user inputs an approval command through the input. The workstation determines the detector corresponding to the acquired image as the detector appropriately mounted at the capturing position input by the user (1036). In addition, a display unit continuously displays the selected image.

On the contrary, if the displayed image is not the image requested by the user (No in 1034), the user inputs a disapproval command through the input, and the workstation displays the image acquired from the detector other than the previously displayed image through the display (1038). The user checks the image acquired by the other detector, and if the image acquired by the other detector is an image requested by the user (YES in 1034), the user inputs an approval command through the input. In response, the workstation determines the other detector as the detector appropriately mounted at the capturing position input by the user (1036). If the user inputs a disapproval command through the input unit, the workstation may display an image acquired by another detector through the display unit (1038).

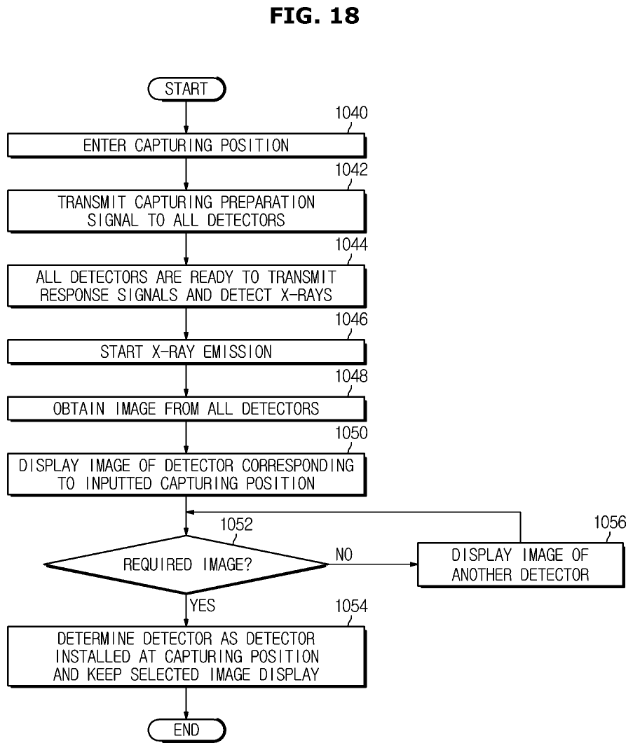

FIG. 18 is a flowchart illustrating another embodiment of a method for position determining of a detector.

Referring to FIG. 18, first, a user may input a photographing position by operating an input unit provided at a workstation of an X-ray imaging apparatus (1040).

When a capturing position is input, the X-ray source of the X-ray imaging apparatus is adjusted to an emission position and/or emission direction according to the input capturing position, and a capturing preparation signal is transmitted from the workstation to a plurality of detectors (all detectors) of the X-ray imaging apparatus (1042).

The plurality of detectors receives a capturing preparation signal and enters a preparation state for detecting X-rays in response to the capturing preparation signal (1044).

Accordingly, the X-ray source starts X-ray emission (1046).

In response to the X-ray source starting the X-ray emission operation, the plurality of detectors respectively perform an operation of reading an electrical signal, and the readout electrical signal is transmitted to the workstation so that the workstation may acquire a plurality of images corresponding to each of the plurality of detectors (1048).

Meanwhile, while the above-described steps 1040 to 1050 are performed, the plurality of detectors may further transmit information about the slope of each detector to the workstation.

The workstation may determine an install position of the plurality of detectors based on the slope transmitted from each of the plurality of detectors. For example, when the tilt of one detector is 0 degrees, the workstation may determine that the one detector is mounted on a table. For another example, when the tilt of one detector is 90 degrees, the workstation may determine that either detector is mounted on a stand. The workstation compares the mounting position of the detector determined based on a pre-input shooting position and the tilt, and determines the detector corresponding to the pre-input shooting position as the detector on which the X-ray is received. In operation 1050, the workstation displays an image acquired from the detector corresponding to the input photographing position to the user through a display unit (1050). In other words, an image corresponding to the input photographing position is displayed to the user.

The user may determine whether the image corresponding to the input capturing position is an intended image in response to an obtained display of the image (1052).

If the displayed image is the image intended by the user (YES in 1052), the user inputs an approval command through the input. In response to the input of the approval command, the workstation determines the detector which outputs the image corresponding to the input capturing position as the detector appropriately mounted at the capturing position input by the user (1054).

On the contrary, if the displayed image is not the image requested by the user (NO in 1052), the user inputs a disapproval command through the input. In response to the input of the disapproval command, the workstation may display an image acquired by a detector other than the image acquired by the detector outputting an image corresponding to the capturing position input through the display (1056). As described above, the user may input an approval or disapproval command for an image acquired by another detector (1052). Accordingly, the display unit of the workstation maintains the image that was previously displayed (1054) or displays the image acquired by another detector (1056).

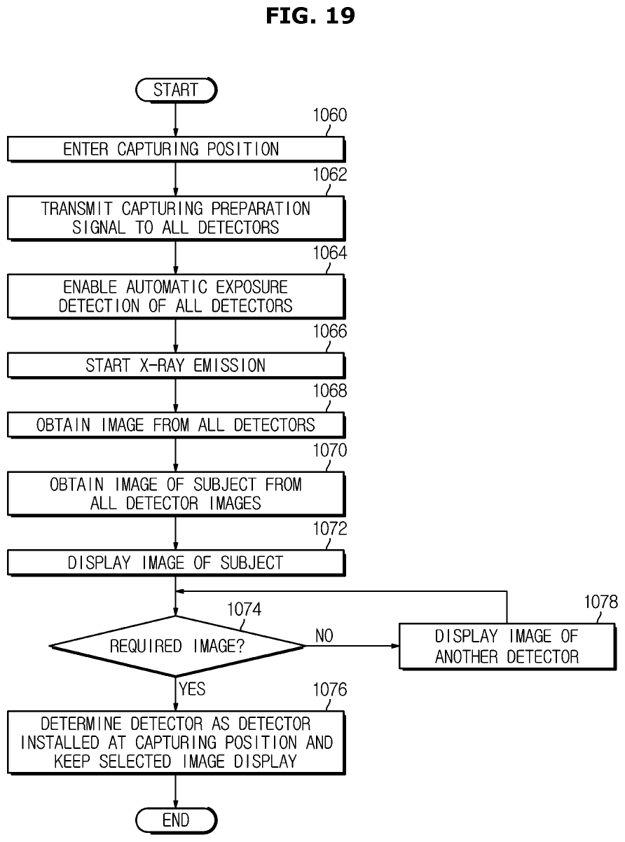

FIG. 19 is a flowchart illustrating another embodiment of a method for position determining of a detector.

As shown in FIG. 19, a user first inputs a capturing position by operating an input provided at a workstation of an X-ray imaging apparatus (1060), and changes the X-ray source to move to an appropriate emission position in response to the capturing position, and/or changes an emission direction as appropriate. In addition, an imaging preparation signal is transmitted from the workstation to a plurality of detectors (which may include all detectors) of the X-ray imaging apparatus (1062).

The plurality of detectors activates an automatic exposure detection operation in response to the reception of the imaging preparation signal (1064). That is, a detector sensor provided in each of the plurality of detectors is activated, and automatically receives the X-rays.

The X-ray source initiates emission of X-rays (1066), and when the X-ray source starts an X-ray emission operation, the plurality of detectors respectively perform an operation of reading an electrical signal. Accordingly, the workstation may obtain a plurality of images from each of the plurality of detectors (1068).

The workstation may detect and acquire an image in which a subject appears among the plurality of images by analyzing or comparing the plurality of images corresponding to each of the plurality of detectors in operation (1070).

When the image in which the subject appears is acquired, the workstation displays the acquired image to the user by using a display in operation (1072).

The user may determine whether the acquired image is an appropriate image in response to the display of the acquired image (1074).

If the displayed image is the image requested by the user (YES in 1074), the user inputs an approval command through the input, and the workstation may determine the detector corresponding to the image acquired in response to the approval command as the detector appropriately mounted at the capturing position input by the user (1076). The selected image continues to be displayed on the display.

On the contrary, if the displayed image is not the image requested by the user (NO in 1074), as described above, the user inputs a disapproval command through the input, and the display of the workstation may display the image acquired by another detector (1078). The user may input an approval or disapproval command for an image acquired by another detector (1074), and according to the approval command or disapproval command, the display may continuously display an image acquired by another detector (1076) or an image acquired by another detector may be displayed (1078).

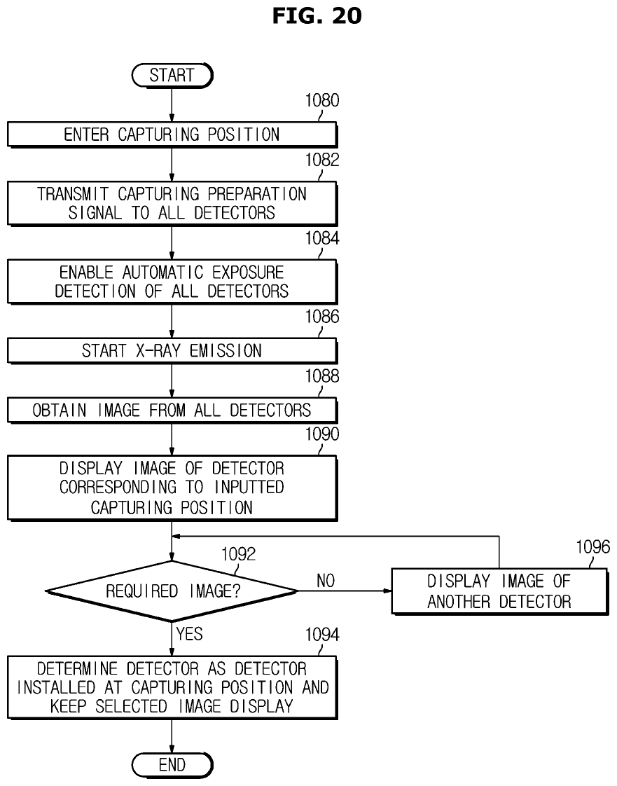

FIG. 20 is a flowchart illustrating another embodiment of a method for position determining of a detector.

As shown in FIG. 20, as described above, a user inputs a capturing position (1080), an emission position and/or an emission direction of an X-ray source is adjusted, and a capturing preparation signal is transmitted to a plurality of detectors (1082).

When the capturing preparation signal is received, the plurality of detectors activates an automatic exposure detection operation in response to the reception of a shooting preparation signal (1084).

After the emission of the X-ray is started (1086), the plurality of detectors respectively output a signal corresponding to an image and transmits it to a workstation (1088).

While the above-described steps 1080 to 1088 are performed, each of the plurality of detectors may transmit information about the slope of each detector to the workstation. As described above, the workstation determines a mounting position of the plurality of detectors based on the inclination transmitted from each of the plurality of detectors, and preferentially displays an image of the detector corresponding to the input photographing position (1090).

The user may determine whether an image corresponding to the input photographing position is an intended image in response to a display of the acquired image (1092).

If the displayed image is the image intended by the user (YES in 1092), the user inputs an approval command through the input, and conversely, if the displayed image is not the image requested by the user (NO in 1092), the user may input a disapproval command through the input.

In response to the input of the approval command, the workstation maintains the display of the image corresponding to the input capturing position. The detector that outputs an image corresponding to the input capturing position may be determined as a detector appropriately mounted at the capturing position input by the user (1094).

In addition, in response to the input of the disapproval command, the workstation may display an image acquired by a detector other than the image obtained by the detector outputting an image corresponding to the input capturing position (1096). As described above, the user may input an approval or disapproval command for an image acquired from another detector (1092). Accordingly, the workstation maintains (1094) the display of the image acquired from the other detector. Alternatively, the image obtained by another detector may be displayed (1096).

A position determining method of the detector according to the above-described embodiment may be implemented in the form of a program that can be driven by a computer device. The program may include program instructions, data files, data structures, and the like, alone or in combination. The program may be designed and produced using machine code or high-level language code. The program may be specially designed to implement the above-described detector position determination method, or may be implemented using various functions or definitions that are well known and available to those skilled in the computer software field. In addition, the computer device may be implemented by including a processor, a memory, or the like that realizes the function of the program, and may further include a communication device as necessary.

The program for implementing the above-described method can be recorded in a computer-readable recording medium. Computer-readable recording media include, for example, magnetic disk storage media such as hard disks or floppy disks, magnetic tapes; optical recording media such as compact disks or DVDs; and magnetic-optical recording media such as floppy disks, and ROMs. It may include a variety of hardware devices capable of storing a specific program to be executed in accordance with the call of a computer, such as a semiconductor storage device such as RAM, or flash memory.

While various embodiments of the X-ray imaging apparatus and the detector position determination method have been described above, the X-ray imaging apparatus and the detector position determination method are not limited to the above-described embodiment. An example of the apparatus and a method for determining the position of various X-ray imaging apparatus or detector that can be modified and modified on the basis of the above-described embodiments by those skilled in the art may also be an example. For example, the techniques described may be performed in an order different than the described method, and/or components of the described systems, structures, devices, circuits, etc. or may be combined in a different form than the described method.

1. An X-ray imaging apparatus, comprising:
an input configured to receive a user command;
an X-ray source configured to radiate X-rays onto a subject according to the user command;
a plurality of detectors respectively outputting a plurality of images in response to performing an X-ray radiation operation of the X-ray source;
a controller configured to determine at least one image among the plurality of images, wherein the at least one image includes an image corresponding to the detector to which the X-ray is estimated to be incident among the plurality of detectors; and
a display configured to display the at least one image.

2. The apparatus of claim 1, wherein the controller detects the image including the subject by analyzing the plurality of images individually, and estimates the detector that transmits a signal for the image including the subject as the detector to which the X-ray is incident.

3. The apparatus of claim 1, wherein the input receives a command for a capturing position of the X-ray, and wherein
the controller obtains information about a direction of each of the plurality of detectors from a corresponding one of the plurality of detectors, and estimates the detector whose direction corresponds to the capturing position of the X-ray as the detector to which the X-ray is incident among the plurality of detectors.

4. The apparatus of claim 1, wherein the controller estimates the detector to which the X-ray is incident based on usage history of each of the plurality of detectors.

5. The apparatus of claim 1, wherein the display displays the plurality of images simultaneously or sequentially,
wherein the input receives a selection command for one of the plurality of images, and
wherein the controller determines the detector to which the X-ray is estimated to be incident from among the plurality of detectors according to the selection command.

6. The apparatus of claim 1, wherein the input receives at least one of an approval command and a disapproval command for the at least one image.

7. The apparatus of claim 6, wherein the display displays another image of the plurality of images instead of the at least one image in response to the input of the disapproval command.

8. The apparatus of claim 6, wherein the input receives a command for a capturing location, and
wherein the controller recognizes the determined detector as the detector corresponding to the capturing location in response to the input of the approval command.

9. The apparatus of claim 1, wherein the plurality of detectors detects whether the X-rays are incident and initiates detection of the X-rays in response to the incidence detection of the X-rays.

10. The apparatus of claim 1, wherein the controller transmits an operation preparation command to the plurality of detectors, and
wherein the plurality of detectors transmits a response signal corresponding to the operation preparation command to the controller in response to receiving the operation preparation command, and prepares for detection of the X-rays.

11. A method for determining an install location of a detector, the method comprising:
radiating X-rays onto a subject by an X-ray source;
outputting a plurality of images in response to performing an X-ray radiation operation of the X-ray source by a plurality of the detectors;
determining at least one image among the plurality of images, wherein the at least one image includes an image corresponding to the detector from which the X-ray is estimated to be incident from the plurality of detectors; and
displaying the at least one determined image.

12. The method of claim 11, wherein the determining of the at least one image among the plurality of images, wherein the at least one image includes the image corresponding to the detector from which the X-ray is estimated to be incident from the plurality of detectors; comprises,
detecting the image including the subject by analyzing the plurality of images individually; and
estimating the detector that transmits a signal for the image including the subject as the detector to which the X-ray is incident.

13. The method of claim 11 further comprising:
receiving a command for a capturing position of the X-ray, and
wherein the determining of the at least one image among the plurality of images, wherein the at least one image includes an image corresponding to the detector from which the X-ray is estimated to be incident from the plurality of detectors; comprises,
obtaining information on a direction of the plurality of detectors from each of the plurality of detectors; and
determining the detector among the plurality of detectors whose direction corresponds to the capturing position of the X-rays.

14. The method of claim 11, wherein the determining of the at least one image among the plurality of images, wherein the at least one image includes an image corresponding to the detector from which the X-ray is estimated to be incident from the plurality of detectors; comprises,
determining the detector to which the X-ray is incident based on usage history of each of the plurality of detectors.

15. The method of claim 11, wherein the determining of the at least one image among the plurality of images, wherein the at least one image includes an image corresponding to the detector from which the X-ray is estimated to be incident from the plurality of detectors; comprises,
displaying the plurality of images simultaneously or sequentially,
receiving a selection command for any one of the plurality of images, and
determining the detector from which the X-ray is estimated to be incident from among the plurality of detectors according to the selection command.
